(19) United States
(12) Patent Application Publication
(10) Pub. No.: US 2025/0114498 A1
(43) Pub. Date: Apr. 10, 2025

(54) Gene Editing and Delivery of Myeloid Cells To Promote Wound Healing

(71) Applicant: The Board of Trustees of the Leland Stanford Junior University, Stanford, CA (US)

(72) Inventors: Geoffrey C. Gurtner, Portola Valley, CA (US); Dominic Henn, Menlo Park, CA (US); Lei S. Qi, Stanford, CA (US); Dehua Zhao, Stanford, CA (US)

(21) Appl. No.: 18/729,844

(22) PCT Filed: Jan. 24, 2023
(86) PCT No.: PCT/US2023/061134

§ 371 Date: Jul. 17, 2024

Related U.S. Application Data
(60) Provisional application No. 63/302,893, filed on Jan. 25, 2022.

(51) Int. Cl. A61L 27/38; A61L 27/20; A61L 27/24; A61L 27/52; C08L 3/04; C08L 89/06; C12N 9/22; C12N 15/11

(52) U.S. Cl. CPC A61L 27/383; A61L 27/20; A61L 27/24; A61L 27/52; C08L 3/04; C08L 89/06; C12N 9/22; C12N 15/111; A61L 2300/64; A61L 2430/34; C12N 2310/20

(57) ABSTRACT

The present disclosure provides compositions and methods for enhanced healing of wounds, e.g. cutaneous wounds, by application of a scaffold or matrix, e.g. a hydrogel, comprising an effective dose of edited dendritic cells (DCs). As used herein, edited dendritic cells refers to mammalian, usually human, dendritic cells that have been genetically modified to alter expression of one or more genes involved in wound healing. In some embodiments edited DC are modified to downregulate or substantially eliminate expression of N-myc downregulated gene 2 (Ndrg2) expression, which cells may be referred to as Ndrg2 KO DCs.

### CROSS REFERENCE TO OTHER APPLICATIONS

This application claims the benefit of U.S. Provisional Application No. 63/302,893, filed Jan. 25, 2022, the contents of which are hereby incorporated by reference in its entirety. INCORPORATION BY REFERENCE LISTING PROVIDED AS A TEXT

A sequence listing is provided herewith as a sequence listing xml, “S20-412_STAN-1803WO_Seqlist” created on Jan. 24, 2023 and having a size of 36,307 Bytes. The contents of the sequence listing xml are incorporated by reference herein in their entirety.

### BACKGROUND

In the United States, 6.5 million patients suffer from chronic wounds, which impact nearly 15% of Medicare beneficiaries at an annual cost estimated at $28-$32 billion. Chronic wounds constitute a significant burden to the healthcare system as a whole and on the individual level, causing an immeasurable negative psychosocial impact on affected patients. Treatment approaches like skin grafting, or tissue engineered skin substitutes are essential in clinical practice but require surgical interventions and often fail in the setting of complex wounds in patients with diabetes and peripheral vascular disease (PVD). These conditions lead to a dysfunctional healing response characterized by an impaired ability to regenerate a functional microvasculature through the process of angiogenesis.

For decades, cell-based therapies have been under development and demonstrated secretory, immunomodulatory, and regenerative effects on chronic wounds. However, unlike in oncology, treatment of chronic wounds with the body's own cells has not been successfully translated into clinical practice, let alone become the standard of care for complex wounds. The “workhorse” of current cell therapy approaches in regenerative medicine are mesenchymal stromal cells (MSCs), which, despite their beneficial effect on wound healing, have a number of disadvantages. Isolation of MSCs requires tissue biopsies or surgical interventions such as liposuction, which are not indicated in the vast majority of patients presenting to wound clinics, and moreover, stem cell cultivation and expansion come at significant costs.

Dendritic cells (DCs) are hematopoietic cells that critically regulate the innate and adaptive immune response. Depending on their activation status, DCs can promote peripheral immune tolerance (tolerogenic DCs), thus limiting the activation of the immune system and tissue damage. For clinical applications, DCs can be easily isolated from the peripheral blood in large quantities using established good manufacturing practice (GMP) protocols for leukaphereses. Multiple clinical trials have reported beneficial effects of tolerogenic DC therapy in the treatment of autoimmune diseases, such as multiple sclerosis, rheumatoid arthritis, type 1 diabetes, Crohn's disease as well as transplant rejection. In addition, Sipuleucil-T, a DC vaccine, has demonstrated survival benefits in patients with castration-resistant prostate cancer, leading to its approval by the US Food and Drug Administration (FDA) in 2010.

Described herein are compositions and methods for the treatment of cutaneous wounds using edited DCs.

### SUMMARY OF THE INVENTION

The present disclosure provides compositions and methods for enhanced healing of wounds, e.g. cutaneous wounds, by application of a scaffold or matrix, e.g. a hydrogel, comprising an effective dose of edited dendritic cells (DCs). As used herein, edited dendritic cells refers to mammalian, usually human, dendritic cells that have been genetically modified to alter expression of one or more genes involved in wound healing. In some embodiments edited DC are modified to downregulate or substantially eliminate expression of N-myc downregulated gene 2 (Ndrg2) expression, which cells may be referred to as Ndrg2 KO DCs. The compositions of the invention find use in treating cutaneous wounds, including chronic wounds, e.g. wounds in diabetic patients or other patients with impaired wound healing. The dose of edited DC is sufficient to enhance the rate of healing, and may provide for an improvement in healing compared to a non-edited DC. The cells can be autologous or allogeneic with the recipient.

As disclosed herein, single-cell RNA sequencing (scRNA-seq) of tolerogenic DCs was used to identify genes that promote a tolerogenic or immature cell state with pro-angiogenic and regenerative capacity. Ndrg2 was identified as marking a specific population of DC progenitors. Ndrg2 was knocked out (KO) using a CRISPR/Cas9 based approach that allowed for streamlined, robust and high-throughput editing of large quantities of DCs, resulting in >90% KO rates in primary DCs with minimal off-target effects. Ndrg2 KO in DCs resulted in a preservation of an immature cell state that was pro-angiogenic. Application of Ndrg2 KO DCs to cutaneous wounds enhanced wound healing in both normal and diabetic wound models.

In some embodiments, Ndrg2 KO DCs are characterized as having reduced or no expression of a Ndrg2 gene or gene product. In some embodiments, the reduction in Ndrg2 expression in DCs results in increased expression of one or more of genes involved in wound healing including, without limitation, of Vascular endothelial growth factor (Vegfa), Fibronectin-1 (Fn1), Matrix metalloproteinase-12 (Mmp12), Peroxiredoxin 4 (Prdx4), Microsomal Glutathione S-Transferase 1 (Mgst1), etc. In some embodiments, the reduction in Ndrg2 expression in DCs results in the reduction in the expression of one or more pro-inflammatory genes including, without limitation, S100 Calcium Binding Protein A6 (S100a6), S100 Calcium Binding Protein A4 (s100a4), etc.

In the methods of the invention, a wound, e.g. a chronic cutaneous wound, is contacted at the surface with a scaffold or matrix, e.g. a hydrogel, comprising an effective dose of edited DCs. The effective dose may be from about 10³ cells/mm² of hydrogel, up to about 10⁶ cells/mm² of hydrogel or more, and may be present at a concentration of from about 10⁴ to about 10¹ cells/mm² of hydrogel. The wound dressing may comprise a support or covering over the scaffold or matrix to provide a barrier. The dressing can remain in place until the wound is healed, e.g. for up to about 3, 5, 7, 9, 11, 13, 15, 17 or more days. Alternatively the administration of dressing can be repeated after a suitable period of time, e.g. after about 3, 5, 7, 9, 11, 13, 15, 17 or more days.

Optionally the hydrogel further comprises protein ligands, e.g. protein ligands involved in cell growth, including, without limitation, growth factors, chemokines, cytokines, fibronectin, cell adhesive peptides (RGDS), laminin, and the like.

In some embodiments of the invention, a hydrogel-cell composition is provided, which hydrogel is comprised of a cross-linked pullulan scaffold, for example a hydrogel comprising from about 5% to about 40% pullulan by weight when hydrated; and collagen at a concentration of from about 1% to about 10% of the total dry weight of the hydrogel, excluding cells, assuming that the hydrogel is a planar configuration of from about 0.5 to about 2.5 mm in thickness. The cell population may be at least about 50% the desired cell type, e.g. at least about 50%; at least about 60% the desired cell type; at least about 70% the desired cell type; at least about 80% the desired cell type; at least about 90% the desired cell type; at least about 95% the desired cell type. The hydrogel may comprise pores of controlled size, usually pores of from about 10-100 m in diameter. Dispersed in the hydrogel is an effective dose of edited DCs within the scaffold. Optionally the hydrogel is fabricated with salt-induced phase inversion and cross-linking to form a reticular scaffold. This soft pullulan-collagen scaffold displays excellent handling characteristics, durability, and a porous dermal-like ultrastructure, and cells are viably sustained within the scaffold. For example, see U.S. Pat. No. 9,636,362, or published application US20170157296A1, each herein specifically incorporated by reference.

In some embodiments, application of a hydrogel-cell composition to a wound results in an enhancement of wound healing. For instance, application of the hydrogel-cell composition may result in a 10%, 20%, 30%, 40%, 50%, 60%, 70%, 80, % 90%, 100% or more than 100% reduction in the time taken for a wound to heal relative to no treatment, or to treatment with a non-edited DC. In some embodiments, application of the hydrogel-cell composition results in an increase in vascularization in wound tissue relative to no treatment. In some embodiments, application of the hydrogel-cell composition results in an upregulation of one or more genes involved in wound healing in fibroblasts residing in the wound bed including, without limitation, Nerve growth factor receptor (Ngfr), Galectin-1 (Lgals1), lysyl hydroxylase 2 (Plod2), peptidyl prolyl cis/trans isomerase (Ppib), Osteopontin (Spp1), etc.

Another aspect of the present disclose provides a method for producing an edited dendritic cell for use in wound healing, the method comprising contacting a dendritic cell or progenitor thereof with a Cas9/single guide RNA-ribonucleoprotein complex (Cas9-RNP) comprising one or more single guide RNAs (sgRNAs) targeted to a gene involved in wound healing. In some embodiments the gene is Ndrg2. Exemplified sgRNA sequences for this purpose include, without limitation, SEQ ID NOs: 1-6. In some embodiments, the one or more sgRNA sequences comprises a spacer sequence that is more than 12 bps complimentary to the gene-coding sequence of Ndrg2 or its regulatory elements. In some embodiments, multiple sgRNAs are used. For instance, two or more, three or more, four or more, or five or more sgRNAs may be used. In some embodiments, the sgRNA has a sequence selected from SEQ ID NO: 4, 5 and 6. In some embodiments, the Cas9-RNP comprises three sgRNAs having sequences corresponding to SEQ ID NO: 1-3. In some embodiments, an mRNA encoding Cas9 is used. Alternative to Cas nuclease, are, for example, Cas nickase, base editor, prime editor, etc. As an alternative to genome editing, nuclease-deactivated Cas-mediated transcriptional silencing or epigenome silencing including H3K9me3, H3K27me3, and/or DNA methylation may be used. In some cases, CRISPRs other than Cas9 can be used, including any of the Cas9, Cas12, Cas13, Cas14 systems or combinations, for example, Cas12a, Cas12b, Cas12f, etc.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIGS. 1A-1J. a, Single-cell RNA sequencing (scRNA-seq) of vitamin D3 (VD3) stimulated and untreated bone marrow-derived dendritic cells (BM-DCs) (10× Genomics Chromium), cells colored by experimental group. b, cells colored by Seurat clusters. c, Vegfa and Ndrg2 expression projected onto UMAP embedding in untreated and VD3 stimulated cells. d, Subset of cluster 8 showing Ndrg2+ progenitor cells, colored by experimental group. e, Expression of Ndrg2 (red) and Cd34, Itgae, and Flt3 (blue) projected onto UMAP embedding of cluster 8 subset. Right column shows merged expression of Ndrg2 and co-expressed genes. f, Flow cytometry of untreated and VD3 stimulated BM-DCs, gated on CD34, CD103, and CD135. g, Schematic of co-culture experiments using BM-DCs and human umbilical vein endothelial cells (HUVECs). h, Endothelial tube formation after co-culture of endothelial cells together with untreated DCs or VD3 and lipopolysaccharide (LPS) stimulated DCs. DCs were stained with calcein red, ECs were stained with calcein AM (green). Cell nuclei were stained with Hoechst (blue). Bottom row shows binary images created using the Angiogenesis Analyzer. j, Luminex multiplex ELISA showing protein expression of 33 differentially expressed proteins in cell culture media of untreated DCs and of DCs stimulated with either VD3, LPS or VD3 and LPS.

FIGS. 2A-2H. a, Schematic for Cas9 ribonucleoprotein (RNP) nucleofection of primary dendritic cells (DCs). b, schematic of Ndrg2 gene, SEQ ID NO:40. Grey rectangles indicate exon regions within the gene. The sequences of the 3 single-guide RNAs (sgRNAs) targeting Ndrg2 (Ndrg2_1, Ndrg2_2, Ndrg2_3) are indicated. c, Flow cytometry of untreated (wild-type, WT) DCs (negative control), DCs transfected with green fluorescent protein (GFP)-fused deactivated Cas9 (dCas9-GFP), and GFP-mRNA (positive control) indicating a high (93%) transfection efficiency of primary DCs with Cas9 protein. d, Percentage of indels per given fragment size after Cas9 nucleofection of DCs. e, Immunofluorescent staining for Ndrg2 in control and CRISPR-edited (Ndrg2-KO) cells. f, Coverage and reads generated by whole genome sequencing at ultrahigh depth (100×) at the Ndrg2 on-target site for CRISPR-edited (red) and wild-type (blue) cells. The difference in coverage between the groups is denoted in black. Vertical black lines indicate the 3 sgRNA cut sites. g, Circle plots indicating different groups of on-target and h, off-target consequences in CRISPR-edited cells (legend). White lines indicate the numbers of different consequences per group.

FIGS. 3A-3J. a, UMAP embedding of single-cell RNA sequencing (scRNA-seq) data from Ndrg2-KO dendritic cells (DCs), vitamin D3 (VD3) stimulated DCs and untreated cells (control). RNA velocity stream vectors computed with scVelo are projected onto the embedding. Cells colored by experimental group (23,871 cells). b, cells colored by Seurat cluster. Cluster 9 represents Ndrg2+ progenitor cells. c, UMAP embedding split by experimental condition and cells colored by cell density. In each plot cells of the indicated condition are colored in yellow/violet and cells of the other two conditions are colored in grey. e, Violin plots of DC maturation markers. f, UMAP embedding split by experimental condition. Ndrg2+ progenitor cells (cluster 9) colored in red. g, Violin plots of wound healing related markers. h, heatmap of the top 10 differentially expressed genes per cluster, sorted by average log fold-change and ordered by experimental condition. i, over-representation analysis (ORA) of the indicated gene sets (GO-BP) projected on the UMAP embedding. j, Ndrg2-KO cells seeded on collagen-pullulan hydrogels stained with calcein AM. 3D reconstruction of stacked confocal microscopy images.

FIGS. 4A-4J. a, Schematic of cell seeding experiments on excisional wounds in wild-type mice. b, Wounds treated with Ndrg2-KO dendritic cells (DCs) healed significantly faster and were completely re-epithelialized on day 12, 4 days faster than wounds treated with control DCs (underwent electroporation with Cas9-RNP using 3 non-targeting sgRNAs) or blank hydrogels. c, Masson's trichrome staining of explanted wound tissue (day 16) of the three groups. Zoomed-in panels show the regenerated epidermis. d, Relative wound area over time (left) and probability of wound closure (right) in the three groups. e, Comparison of dermal thickness of wound tissue from the three experimental groups. f, Immunofluorescent staining of wound tissue for CD31 marking blood vessels. g, Schematic of cell seeding experiments on excisional wounds in diabetic mice. h, Wounds treated with Ndrg2-KO DCs healed significantly faster and were epithelialized on day 16, 5 days faster than wounds treated with control DCs or blank hydrogels. i, Relative wound area over time in diabetic wound tissue for the three groups. j, Immunofluorescent staining of diabetic wound tissue (day 21) for CD31.

FIGS. 5A-5M. a, Schematic of cell seeding experiments to analyze wound tissue using single-cell RNA sequencing (scRNA-seq). b, UMAP embedding of three experimental conditions, colored by cell type. c, Cells colored by experimental conditions. d, Numbers of differentially expressed genes (DEG) colored by cell type (adjusted p-value<0.05, FC>0.5). e, Volcano plot showing significantly deregulated genes across all cell types. Right half of plot representing cells isolated from wounds treated with Ndrg2-KO dendritic cells (DCs), left half of plot showing cells isolated from control wounds. Cells are colored by cell type as in b and d. f, Subset of fibroblasts for all three conditions. g, Expression of wound healing related genes projected onto fibroblast subset UMAP embedding and as violin plots. h, Heatmaps showing the expression of outgoing signaling pathways in Ndrg2-KO dendritic cells (DCs, left) and control DCs (right) that target wound cells (integrated scRNA-seq data of FIGS. 3 and 7). i, Interaction strength with wound cells across differentially regulated signaling pathways for Ndrg2KO-DCs (top) and control cells (bottom). j, VEGF signaling between Ndrg2KO DCs and fibroblasts (not significant for control DCs). k, IGF signaling in wounds treated with Ndrg2-KO DCs (left) or control DCs (right). l, PDGF signaling in wounds treated with Ndrg2-KO DCs (left) or control DCs (right). m, SPP1 signaling in wounds treated with Ndrg2-KO DCs (left) or control DCs (right).

FIGS. 6A-6F. a, Expression of markers for mature and immature DCs projected onto the UMAP embedding. b, Dot plot indicating Ndrg2 and Vegfa expression in VD3 treated and control cells. c, Ndrg2 expression in different Seurat clusters of control cells. d, Expression of Ndrg2 (red) and Kit, Csf1r, and Clec9a (blue) projected onto UMAP embedding of cluster 8 subset. Right column shows merged expression of Ndrg2 and co-expressed genes. e, Endothelial cell (EC) tube formation after co-culture of ECs together with dexamethasone stimulated DCs and after EC monoculture in basal media (negative control). f, Zoomed-in image of EC tube formation after co-culture of ECs with VD-stimulated DCs indicating close interaction of DCs and ECs.

FIGS. 7A-7C. a, Optimization of Cas9 RNP approach using sgRNAs from Synthego and IDT at different cell concentrations. b, Alignment between predicted off-target sites and sgRNA sequences, SEQ ID NOs:18-39. Rows with arrows demonstrate a high alignment. Red arrow indicate potential off-target sites that show a protospacer adjacent motif (PAM) sequence. c, Off-target variant classes. White lines indicate the numbers of different consequences per group.

FIGS. 8A-8C. a, Ndrg2 expression projected onto UMAP embedding of in vitro scRNAseq dataset, plot split by experimental group. Zoom-in panels showing cluster 9 (Ndrg2+ progenitors). b, Vegfa expression projected onto UMAP embedding of in vitro scRNAseq dataset, plot split by experimental group. c, Violin plots showing expression of genes (Amy1 and Ftsj1) at potential off-target sites.

FIGS. 9A-9B. a, Masson's trichrome staining of explanted wound tissue from diabetic mice (day 21). b, Probability of wound closure in diabetic mice after treatment with Ndrg2-KO dendritic cells (DCs), control DCs (nucleofection with 3 non-targeting sgRNAs), and blank hydrogels.

### DESCRIPTION OF THE SPECIFIC EMBODIMENTS

Compositions and methods are provided for wound healing. The methods comprise contacting a wound on an individual with a scaffold or matrix dressing, e.g. a hydrogel, comprising an effective dose of genetically edited DCs. In some embodiments the scaffold or matrix is a hydrogel, for example a cross-linked pullulan hydrogel.

The dressings of the invention are suitable for burn patients, diabetic ulcers, venous ulcers, partial- and full-thickness wounds, pressure ulcers, chronic vascular ulcers, trauma wounds, draining wounds, and surgical wounds. The dressing is easily applied onto the wound bed and can remain in place until wound healing is complete, or administration can be repeated as needed.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

Scaffold or matrix. A support that provides for maintaining viability and location of cells when placed over a cutaneous wound.

Hydrogel. Hydrogels are known and used in the art for wound healing. Hydrogels useful in the methods of the invention maintain viability of entrapped cells for a period of time sufficient to enhance wound healing. Typically hydrogels are, by weight, up to about 50%, up to about 55%, up to about 60%, up to about 65%, up to about 70%, up to about 75%, up to about 80%, up to about 85%, up to about 90% water, with the remaining weight comprising a suitable polymer, e.g. pullulan and collagen, glycosaminoglycan, acrylate, 2-hydroxymethyl methacrylate and ethylenedimethacrylate copolymer, carboxymethylcellulose, chitosan, gelatin, etc., or other suitable hydrophilic polymers as known in the art, including carbohydrate based polymers. Hydrogels can swell extensively without changing their gelatinous structure and are available for use as amorphous (without shape) gels and in various application systems, e.g. flat sheet hydrogels and non-woven dressings impregnated with amorphous hydrogel solution. Flat sheet (film) hydrogel dressings have a stable cross-linked macrostructure and therefore retain their physical form as they absorb fluid.

In some embodiments a cross-linked hydrogel film is fabricated using pullulan and collagen under conditions that provided for cross-linking and pore formation. Collagen is added to a mixture of pullulan, cross-linking agent and pore-forming agent (porogen), where the collagen is provided at a concentration of at least about 1%, and not more than about 12.5% relative to the dry weight of the pullulan. Collagen may be provided at a concentration of about 1%, about 2.5%, about 5%, about 7.5%, about 10%, usually at a concentration of from about 2.5% to about 10%, and may be from about 4% to about 6% relative to the dry weight of the pullulan. The collagen is typically a fibrous collagen, e.g. Type I, II, III, etc. Cross-linking agents of interest include sodium trimetaphosphate (STMP) or a combination of or a combination of sodium trimetaphosphate and sodium tripolyphosphate (STMP/STPP). The cross-linking agent can be included in a wt/wt ratio relative to the pullulan of from about 5:1 to about 1:5, and may be about 4:1, 3:1, 2:1, 1.75:1, 1.5:1, 1.25:1, 1:1, 1:1.25, 1:1.5, 1:1.75, 2:1, 3:1, 4:1, etc. Porogens of interest for in-gel crystallization include any suitable salt, e.g. KCl. The porogen can be included in a wt/wt ratio relative to the pullulan of from about 5:1 to about 1:5, and may be about 4:1, 3:1, 2:1, 1.75:1, 1.5:1, 1.25:1, 1:1, 1:1.25, 1:1.5, 1:1.75, 2:1, 3:1, 4:1, etc. The suspension of collagen, pullulan, cross-linker and porogen, in the absence of cells, is poured and compressed to form sheets. Preferred thickness is at least about 1 mm and not more than about 5 mm, usually not more than about 3 mm, and may be from about 1 to 2.5 mm, e.g. about 1.25, 1.5, 1.75, 2 mm thick. Pores are formed in the hydrogel through rapid dessication of swollen hydrogels by phase inversion. Dehydration results in localized super-saturation and crystallization of the porogen. Pullulan and collagen are forced to organize around the crystals in an interconnected network, which results in reticular scaffold formation following KCl dissolution.

The films may be stored in a dried state, and are readily rehydrated in any suitable aqueous medium. The aqueous nature of hydrogel substrates provides an ideal environment for cellular growth and sustainability.

Mechanical features of the hydrogel include average pore size and scaffold porosity. Both variables vary with the concentration of collagen that is present in the hydrogel. For a hydrogel comprising 5% collagen, the average pore size will usually range from about 25 μm to about 50 μm, from about 30 μm to about 40 μm, and may be about 35 μm. For a hydrogel comprising 10% collagen the average pore size will usually range from about 10 μm to about 25 μm, from about 12 μm to about 18 μm, and may be about 15 μm. One of skill in the art will readily determine suitable hydrogels at other collagen concentrations. The scaffold porosity will usually range from about 50% to about 85%, and may range from about 70% to about 75%, and will decrease with increasing concentrations of collagen. Hydrogels lacking collagen do not display any birefringence with polarizing light microscopy, while the hydrogels comprising collagen are diffusely birefringent.

Pullulan. A polysaccharide produced by the fungus Aureobasidium pullulans. It is a linear homopolysaccharide consisting of alpha-(1-6) linked maltotriose units and exhibits water retention capabilities in a hydrogel state which makes it an ideal therapeutic vehicle for both cells and biomolecules. Additionally, pullulan contains multiple functional groups that permit cross-linking and delivery of genetic material and therapeutic cytokines. Furthermore, pullulan-based scaffolds have been shown to enhance both endothelial cell and smooth muscle cell behavior in vitro.

Collagen. As used herein the term “collagen” refers to compositions in which at least about 50%, at least about 60%, at least about 70%, at least about 80%, at least about 90%, at least about 95% or more of the protein present is collagen in a triple helical configuration. Collagens are widely found in vertebrate species, and have been sequenced for many different species. Due to the high degree of sequence similarity between species, collagen from different species can be used for biomedical purposes, e.g. between mammalian species. Typical commercial animal sources include the bovine Achilles tendon, calfskin and the bones of cattle. In some embodiments the collagen used in the preparation of the oriented thin film is Type I, Type II, or Type III collagen, and is derived from any convenient source, e.g. bovine, porcine, etc., usually a mammalian source.

Collagen has a triple-stranded rope-like coiled structure. The major collagen of skin, tendon, and bone is collagen I, containing 2 alpha-1 polypeptide chains and 1 alpha-2 chain. The collagen of cartilage contains only 1 type of polypeptide chain, alpha-1. The fetus also contains collagen of distinctive structure. The genes for types I, II, and III collagens, the interstitial collagens, exhibit an unusual and characteristic structure of a large number of relatively small exons (54 and 108 bp) at evolutionarily conserved positions along the length of the triple helical gly-X-Y portion.

Types of collagen include I (COL1A1, COL1A2); II (COL2A1); III (COL3A1); IV (COL4A1, COL4A2, COL4A3, COL4A4, COL4A5, COL4A6); V (COL5A1, COL5A2, COL5A3); VI (COL6A1, COL6A2, COL6A3); VII (COL7A1); VIII (COL8A1, COL8A2); IX (COL9A1, COL9A2, COL9A3); X (COL10A1); XI (COL11 A1, COL11A2); XII (COL12A1); XIII (COL13A1); XIV (COL14A1); XV (COL15A1); XVI (COL16A1); XVII (COL17A1); XVIII (COL18A1); XIX (COL19A1); XX (COL20A1); XXI (COL21A1); XXII (COL22A1); XXIII (COL23A1); XXIV (COL24A1); XXV (COL25A1); XXVII (COL27A1); XXVIII (COL28A1). It will be understood by one of skill in the art that other collagens, including mammalian collagens, e.g. bovine, porcine, equine, etc. collagen, are equally suitable for the methods of the invention.

Supports. A variety of solid supports or substrates may be used with the scaffold or matrix, e.g. a hydrogel, including deformable supports or barriers. By deformable is meant that the support is capable of being damaged by contact with a rigid instrument. Examples of deformable solid supports include polyacrylamide, nylon, nitrocellulose, polypropylene, polyester films, such as polyethylene terephthalate; PDMS (polydimethylsiloxane); tagamet; etc. as known in the art for the fabrication of wound dressings.

Cells. The films of the invention provide a substrate for maintenance of dendritic cells, which are typically mammalian cells, where the term refers to any animal classified as a mammal, including humans, domestic and farm animals, and zoo, laboratory, sports, or pet animals, such as dogs, horses, cats, cows, mice, rats, rabbits, etc. Preferably, the mammal is human. The cells which are employed may be fresh, frozen, or have been subject to prior culture. The cells may be autologous or allogeneic relative to the intended recipient. Cells can be obtained from a blood draw, e.g. allogeneic or autologous, or may be differentiated in vitro from a stem or progenitor cell population, which include without limitation induced pluripotent stem cells, embryonic stem cells, hematopoietic stem cells, and the like.

Dendritic cell (DC) refers to any member of a diverse population of morphologically similar cell types found in lymphoid or non-lymphoid tissues. DCs are referred to as “professional” antigen presenting cells, and have a high capacity for sensitizing MHC-restricted T cells. DCs may be recognized by function, by phenotype and/or by gene expression pattern, particularly by cell surface phenotype. These cells are characterized by their distinctive morphology, high levels of surface MHC-class II expression and ability to present antigen to CD4⁺ and/or CD8⁺ T cells, particularly to naive T cells (Steinman et al. (1991) Ann. Rev. Immunol. 9:271; incorporated herein by reference for its description of such cells).

Immature DCs express low levels of MHC class II, but are capable of endocytosing antigenic proteins and processing them for presentation in a complex with MHC class II molecules. Activated DCs express high levels of MHC class II, ICAM-1 and CD86, and are capable of stimulating the proliferation of naïve allogeneic T cells, e.g. in a mixed leukocyte reaction (MLR). Functionally, DCs may be identified by any convenient assay for determination of antigen presentation. Such assays may include testing the ability to stimulate antigen-primed and/or naïve T cells by presentation of a test antigen, followed by determination of T cell proliferation, release of IL-2, and the like.

Typical protocols for in vitro differentiation of dendritic cells from monocytes are described, for example, in Davies and Gordon, Methods in Molecular Biology vol. 290: Basic Cell Culture Protocols, Third edition; and Zhang et al. (2008) Curr. Protoc. Immunology November, each herein specifically incorporated by reference. Such methods generally utilize mononuclear cells from peripheral blood (PBMC), which can be enriched by selection for cells bearing monocyte markers or by adherence, for example to a culture dish, although such enrichment is not required. Markers for dendritic cells include CD11c, CD11 b and MHC-II.

For example, a sample of PBMC can be obtained from a patient or from a suitable donor. Cells are cultured in the presence of suitable factors, e.g. GM-CSF. Adherent cells are discarded, and DC in suspension are used for gene editing.

It will be understood by those of skill in the art that expression levels reflect detectable amounts of the marker (e.g., protein or nucleic acid) on and/or in the cell. A cell that is negative for staining (e.g., the level of binding of a marker specific reagent is not detectably different from a matched control) may still express minor amounts of the marker. And while it is commonplace in the art to refer to cells as “high”, “+”, “positive”, “low”, “−”, or “negative” for a particular marker, actual expression levels are quantitative traits. For example, number of detected molecules can vary by several logs, yet still be characterized as “positive”. When a protein marker is used, the staining intensity (e.g., of a marker-specific antibody) can be monitored by any method suitable for assaying protein expression, e.g., flow cytometry, Western blotting, mass spectrometry, and enzyme-linked immunosorbent assay (ELISA).

As one example, in flow cytometry, lasers detect the quantitative levels of fluorochrome (which is proportional to the amount of cell marker bound by specific reagents, e.g. antibodies). Flow cytometry, or FACS, can also be used to separate cell populations based on the intensity of binding to a specific reagent (or combination of reagents), as well as other parameters such as cell size and light scatter. Although the absolute level of staining may differ with a particular fluorochrome and reagent preparation, the data can be normalized to a control. As such, a population of cells can be enriched for macrophages by using flow cytometry to sort and collect those cells (e.g., only those cells) with the desired profile.

In order to normalize the distribution to a control, each cell is recorded as a data point having a particular intensity of staining. These data points may be displayed according to a log scale, where the unit of measure is arbitrary staining intensity. In one example, the brightest stained cells in a sample can be as much as 4 logs more intense than unstained cells. When displayed in this manner, it is clear that the cells falling in the highest log of staining intensity are bright, while those in the lowest intensity are negative. The “low” positively stained cells have a level of staining brighter than that of an isotype matched control, but is not as intense as the most brightly staining cells normally found in the population. An alternative control may utilize a substrate having a defined density of marker on its surface, for example a fabricated bead or cell line, which provides the positive control for intensity.

Regenerative factors. Polypeptide growth factors and cell-signaling molecules may be included in a hydrogel film. Protein ligands can be printed on the hydrogels by precise micro-contact printing methods. Alternatively the proteins may be included in the initial fabrication of the matrix. Polypeptides of interest as growth factors include, without limitation, the following molecules, where one or more of the factors may be patterned on a matrix. The native form of the polypeptides may be used, or variants thereof, e.g. truncated versions that maintain biological activity; stabilized variants; conjugated engineered for improved adhesion to the hydrogel matrix, and the like.

Platelet-derived growth factor (PDGF) is a family of potent activators for cells of mesenchymal origin, and a stimulator of chemotaxis, proliferation and new gene expression in monocytes, macrophages and fibroblasts, accelerating ECM deposition. This family of growth factors exists in both homo- and heterodimeric forms.

Cytokines of the transforming growth factor-β family (TGF-β) are multifunctional regulators of cell growth, differentiation and ECM formation. In mammals, there are three isoforms, TGF-β1, TGF-β2 and TGF-β3. In particular, in relation to wound healing in the skin, TGF-β1 and TGF-β2 are implicated in cutaneous scarring, whereas TGF-β3 is known to have an anti-scarring effect.

Bone morphogenetic proteins (BMPs) are members of the TGF-β superfamily. There are 15 members and although they are known for their role in bone and cartilage formation, they have diverse roles in many other developmental processes.

Fibroblast growth factors (FGFs) are a family of 21 isoforms with a broad spectrum of activities, including regulation of cell proliferation, differentiation and migration. FGFs 1, 2, 5, 7 and 10 are upregulated during adult cutaneous wound healing. bFGF may have the ability to accelerate tissue regeneration in artificial dermis.

Vascular endothelial growth factor (VEGF) is induced during the initial phase of skin grafting, where endogenous fibrin clots are known to form a provisional matrix and to promote angiogenesis. Growth factors such as VEGF increase in such wounds to stimulate angiogenesis.

Epidermal growth factor (EGF) has been implicated in wound healing and homeostasis in a number of tissues.

Hepatocyte growth factor/scatter factor (HGF/SF) is a pleiotrophic growth factor produced principally by cells of mesenchymal origin. HGF has been implicated in enhancing the cutaneous wound healing processes of re-epithelialization, neovascularization and granulation tissue formation.

Antimicrobial agents. The hydrogels may further comprise antimicrobial agents. Agents of interest include a wide variety of antibiotics, as known in the art. Classes of antibiotics include penicillins, e.g. penicillin G, penicillin V, methicillin, oxacillin, carbenicillin, nafcillin, ampicillin, etc.; penicillins in combination with P lactamase inhibitors, cephalosporins, e.g. cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc. Antiviral agents, e.g. acyclovir, gancyclovir, etc. may also be included.

Wound dressing. films of the invention find use as a wound dressing, or artificial skin, by providing an improved substrate that minimizes scarring. An effective bioactive wound dressing can facilitate the repair of wounds that may require restoration of both the epidermis and dermis. For example, a hydrogel thin film is placed onto, and accepted by, the debrided wound of the recipient and provide a means for the permanent re-establishment of the dermal and epidermal components of skin. The graft suppresses the formation of granulation tissue which causes scarring.

Additional criteria for biologically active wound dressings include: rapid adherence to the wound soon after placement; proper vapor transmission to control evaporative fluid loss from the wound and to avoid the collection of exudate between the wound and the dressing material. Skin substitutes should act as barrier to microorganisms, limit the growth of microorganisms already present in the wound, be flexible, durable and resistant to tearing. The substitute should exhibit tissue compatibility, that is, it should not provoke inflammation or foreign body reaction in the wound which may lead to the formation of granulation tissue. An inner surface structure of a hydrogel thin film is provided that permits ingrowth of fibro-vascular tissue. An outer surface structure may be provided to minimize fluid transmission and promote epithelialization.

Typical bioabsorbable materials for use in the fabrication of porous wound dressings, skin substitutes and the like, include synthetic bioabsorbable polymers such as polylactic acid or polyglycolic acid, and also, biopolymers such as the structural proteins and polysaccharides. The finished dressing prior to cell seeding is packaged and preferably radiation sterilized. Such biologically active products can be used in many different applications that require the regeneration of dermal tissues, including the repair of injured skin and difficult-to-heal wounds, such as burn wounds, venous stasis ulcers, diabetic ulcers, etc.

A CRISPR/Cas protein (also referred to herein as a CRISPR/Cas endonuclease) interacts with (binds to) a corresponding guide RNA to form a ribonucleoprotein (RNP) complex (referred to herein as a CRISPR/Cas complex) that is targeted to a particular site (a target sequence) in a target genome via base pairing between the guide RNA and a target sequence within the target genome. A guide RNA includes (i) a nucleotide sequence (a guide sequence) that is complementary to a sequence (the target site) of a target DNA and (ii) a protein-binding region that includes a double stranded RNA (dsRNA) duplex and bind to a corresponding CRISPR/Cas protein. The guide RNA can be readily modified in order to target any desired sequence within a target genome (by modifying the guide sequence). A wild type CRISPR/Cas protein (e.g., a Cas9 protein) normally has nuclease activity that cleaves a target nucleic acid (e.g., a double stranded DNA (dsDNA)) at a target site defined by the region of complementarity between the guide sequence of the guide RNA and the target nucleic acid. The term “CRISPR/Cas protein,” as used herein, includes wild type CRISPR/Cas proteins, and also variant CRISPR/Cas proteins, e.g., CRISPR/Cas proteins with one or more mutations in a catalytic domain rendering the protein a nickase.

A target DNA (e.g., genomic DNA) that can be recognized and cleaved by a CRISPR/Cas protein (e.g., Cas9) is a DNA polynucleotide that comprises a “target site” or “target sequence.” The terms “CRISPR/Cas target site” or “CRISPR/Cas target sequence” are used interchangeably herein to refer to a nucleic acid sequence present in a target DNA (e.g., genomic DNA of a cell) to which a CRISPR/Cas guide RNA can bind, allowing cleavage of the target DNA by the CRISPR/Cas endonuclease. The strand of the target DNA that is complementary to and hybridizes with the CRISPR/Cas guide RNA is referred to as the “complementary strand” or the “target strand’ and the strand of the target DNA that is complementary to the “complementary strand” (and is therefore not complementary to the guide RNA) is referred to as the “non-complementary strand” or “non-target strand.” A target sequence can be any desired length and, in some cases, can depend upon the type of CRISPR/Cas guide RNA and CRISPR/Cas protein that will be used to target the target sequence.

A wild type CRISPR/Cas protein (e.g., Cas9 protein) normally has nuclease activity that cleaves a target nucleic acid (e.g., a double stranded DNA (dsDNA)) at a target site defined by the region of complementarity between the guide sequence of the guide RNA and the target nucleic acid. In some cases, site-specific targeting to the target nucleic acid occurs at locations determined by both (i) base-pairing complementarity between the guide nucleic acid and the target nucleic acid; and (ii) a short motif referred to as the “protospacer adjacent motif” (PAM) in the target nucleic acid. For example, when a Cas9 protein binds to (in some cases cleaves) a dsDNA target nucleic acid, the PAM sequence that is recognized (bound) by the Cas9 polypeptide is present on the non-complementary strand (the strand that does not hybridize with the targeting segment of the guide nucleic acid) of the target DNA. CRISPR/Cas (e.g., Cas9) proteins from different species can have different PAM sequence requirements.

A nucleic acid that binds to a class 2 CRISPR/Cas endonuclease (e.g., a Cas9 protein; a type V or type VI CRISPR/Cas protein; a Cpf1 protein; etc.) and targets the complex to a specific location within a target nucleic acid is referred to herein as a “guide RNA” or “CRISPR/Cas guide nucleic acid” or “CRISPR/Cas guide RNA.” A guide RNA provides target specificity to the complex (the RNP complex) by including a targeting segment, which includes a guide sequence (also referred to herein as a targeting sequence), which is a nucleotide sequence that is complementary to a sequence of a target nucleic acid.

As used herein, “CRISPR/Cas9 system” typically includes a polynucleotide sequence (which may, together, be referred to as an expression cassette, or one or more sgRNAs may be separately encoded and referred to as a cassette), wherein the polynucleotide sequence encodes the Cas9 nuclease alone, or also encodes one or more single guide RNAs (sgRNAs) as well as the Cas9 nuclease. In some instances, the expression cassette encoding the CRISPR/Cas9 system is referred to as a “transgene.”

Edited Dendritic cells. As used herein, edited dendritic cells refers to mammalian, usually human, dendritic cells that have been genetically modified to alter expression of one or more genes involved in wound healing. In some embodiments edited DC are modified to downregulate or substantially eliminate expression of N-myc downregulated gene 2 (Ndrg2) expression, which cells may be referred to as Ndrg2 KO DCs. The cells can be modified by in vitro programmed genome editing, e.g. with a CRISPR-based system, before or after culture.

A population of dendritic cells or progenitors thereof are isolated from a suitable source, e.g. a mammal such as a human. Sources may include bone marrow, peripheral blood, lymphoid tissue, and the like. The cells may be cultured in media, for example, supplemented with an effective dose of GM-CSF. After culture, e.g. after about 3 to about 10 days in culture, the adherent cells can be separated from cells in suspension, which represent the DC fraction of the cultures.

The method includes introducing single-guide RNAs (sgRNAs) into the cell population, and may comprise simultaneous introduction of multiple guide RNAs into the cell population. The guide RNAs (sgRNAs) include nucleotide sequences that are complementary to the target chromosomal DNA. The sgRNAs can be, for example, engineered single chain guide RNAs that comprise a crRNA sequence (complementary to the target DNA sequence) and a common tracrRNA sequence, or as crRNA-tracrRNA hybrids. The sgRNAs can be introduced into the cell or the organism as a DNA (with an appropriate promoter), as an in vitro transcribed RNA, or as a synthesized RNA. RNPs can be assembled using Cas9 Nuclease and directly synthesized together with from one to three guide RNAs. The RNPs can be electroporated into the cells.

### Devices and Methods

Devices are described here for accelerating wound healing and/or ameliorating the formation of scars and/or keloids at a wound site. The scars may be any type of scar, e.g., a normal scar, a hypertrophic scar, etc. In general, the devices are configured to be removably secured to a skin surface near a wound. The devices of the invention comprise a scaffold or matrix, e.g. a hydrogel film seeded with an effective dose of genetically edited DCs. Usually the film is seeded with cells prior to use. The hydrogel may be applied to the wound immediately after seeding or after culturing cells in the hydrogel for about 1 to about 24 hours. The film may optionally comprise regenerative protein factors, as described herein, which protein factors may be specifically patterned on the film, or may be integrated in the film, or otherwise coupled to the film. A diverse array of active agents or ingredients may be present in the compositions, as described above. Depending on the nature of the agent, the amount of active agent present in the composition may ranges from about 0.2 to 10%, e.g., from about 0.2 to 5%, e.g., from about 0.5 to 5%. The pH of the patch compositions typically is one that lies in a physiologically acceptable range, where the pH typically ranges from about 3.0 to 8.0 and more typically ranges from about 4.0 to 7.0.

The wound dressing may be attached or adhered to a substrate, e.g. a breathable protective layer, or other protective layer. Alternatively the cell-containing dressing may be separately configured from a protective dressing. In certain embodiments, a cell-containing dressing composition may be present on a support or backing. The support is generally made of a flexible material which is capable of fitting in the movement of the human body and includes, for example, various non-woven fabrics, woven fabrics, spandex, flannel, or a laminate of these materials with polyethylene film, polyethylene glycol terephthalate film, polyvinyl chloride film, ethylene-vinyl acetate copolymer film, polyurethane film, and the like. By “flexible” it is meant that the support may be substantially bent or folded without breaking, tearing, ripping, etc. The support may be porous or non-porous, but is typically non-porous or impermeable to the hydrogel composition, active agent if employed and fluids, e.g., any fluids exuded from the wound site.

The length and width dimensions of the support are typically substantially commensurate, including exactly commensurate, with the length and width dimensions of the hydrogel patch composition with which it is associated. The support layer typically may have a thickness that ranges from about 10 m to about 1000 μm, but may be less than about 10 m and/or greater than 1000 m in certain embodiments.

In addition to the scaffold or matrix, e.g. hydrogel patch composition and the optional support layer, the subject patches may also include a release film on the surface of the hydrogel composition layer opposite the backing that provides for protection of the hydrogel composition layer from the environment. The release film may be any convenient material, where representative release films include polyesters, such as PET or PP, and the like.

The shape of the dressing may vary, where representative shapes include square, rectangle, oval, circle, triangular, etc. The size of the dressing may also vary, where in many embodiments the size ranges from about 1 cm² or less to about 1000 cm² or more, e.g., in certain embodiments ranges from about 10 to about 300 cm², e.g., from about 20 to about 200 cm², e.g., about 130 cm² to about 150 cm². In certain embodiments, the surface area is sufficient to cover a substantial portion or even the entire truck or even a substantial portion of the entire body or even the entire body of a subject. Accordingly, the surface area may range from about 1000 cm² to about 5000 cm² or more. It should be noted that the above manufacturing protocol is merely representative. Any convenient protocol that is capable of producing the subject compositions, as described above, may be employed.

The subject methods find use in any application in which the treatment of a wound of a subject is desired. Generally, such subjects are “mammals” or “mammalian”, where these terms are used broadly to describe organisms which are within the class mammalia, including the order carnivore (e.g., dogs and cats), rodentia (e.g., mice, guinea pigs, and rats), and primates (e.g., humans, chimpanzees, and monkeys). In many embodiments, the subject is a human.

Accordingly, the subject methods may be used to treat a wide variety of open- and closed-skin wounds such that the subject methods may be used to treat wounds that have resulted from a variety of causes, e.g., as a result of a condition such as a disease state, a physical injury such as a fall, scrape, stab wound, gun shot, surgical wound, infection, burn wounds etc., wartime injuries such as bombs, bullets, shrapnel. In addition, the subject methods may be used in conjunction with other reconstructive surgical techniques to treat wounds, such as skin grafts or autologous flaps. Likewise, the subject methods may treat wounds of various dimensions. For example, the subject methods may be employed to with both deep tissue wounds and shallow or superficial wounds, where certain wounds may have depths that reach the muscle. Wounds may be confined to the epidermis such that they do not penetrate into the dermal layer, may be as deep as the dermis or deeper, e.g., may penetrate to or through the dermis and even to or through the subcutaneous tissue layer or deeper, e.g., may penetrate through or to the muscle layer or further. For example, the subject methods may be used to debride wounds that having a depth that ranges from about 0.005 mm to about 2.35 mm, e.g., from about 0.007 mm to about 2.3 mm, e.g., from about 0.01 mm to about 2 mm.

Types of wounds that may be treated with the subject invention include, but are not limited to, ulcers, including pressure ulcers, diabetic ulcers (e.g., diabetic foot ulcers), venous ulcers, lower leg ulcer, etc.; burns (first, second and third degree burns) including scalds, chemical burns, thermal burns such as flame burns and flash burns, ultraviolet burns, contact burns, radiation burns, electrical burns, etc.; bone infections (osteomyelitis); gangrene; skin tears or lacerations, such as made by knives, etc.; abrasions; punctures such as made by nails, needles, wire, and bullets, etc.; incisions such as made by knives, nails, sharp glass, razors, etc.; avuls; amputations; post-operative infections; surgical wounds; brown recluse spider wounds; failing or compromised skin/muscle grafts or flaps; bites; slash wounds, i.e., a wound where the length is greater than the depth; bruises; and the like, or a combination of one or more of the above.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

Such biologically active products can be used in many different applications that require the regeneration of dermal tissues, including the repair of injured skin and difficult-to-heal wounds, such as burn wounds, venous stasis ulcers, diabetic ulcers, etc.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Experimental

### Example 1

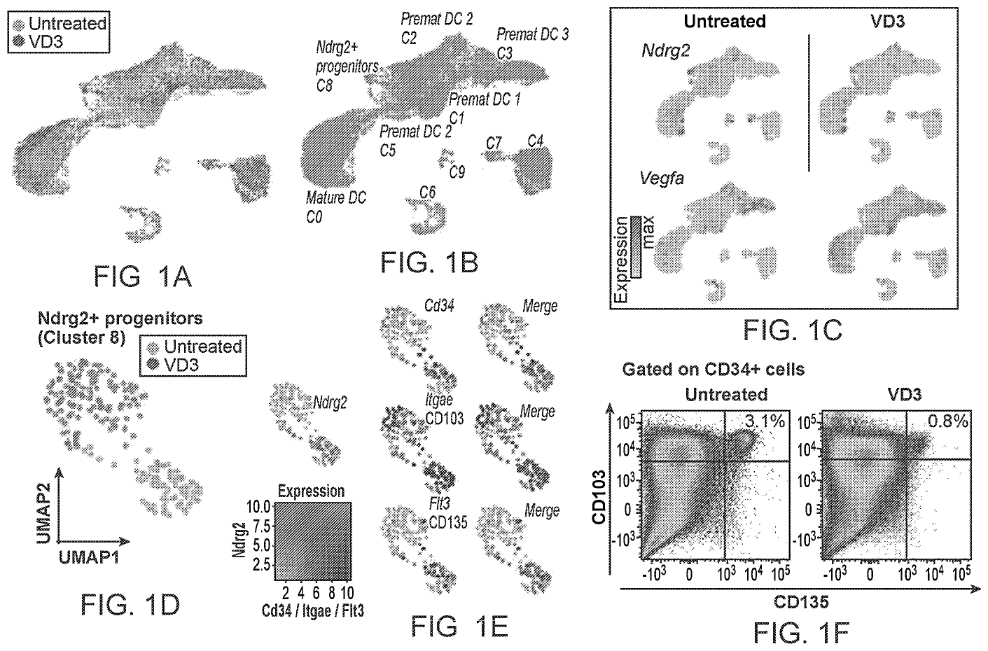

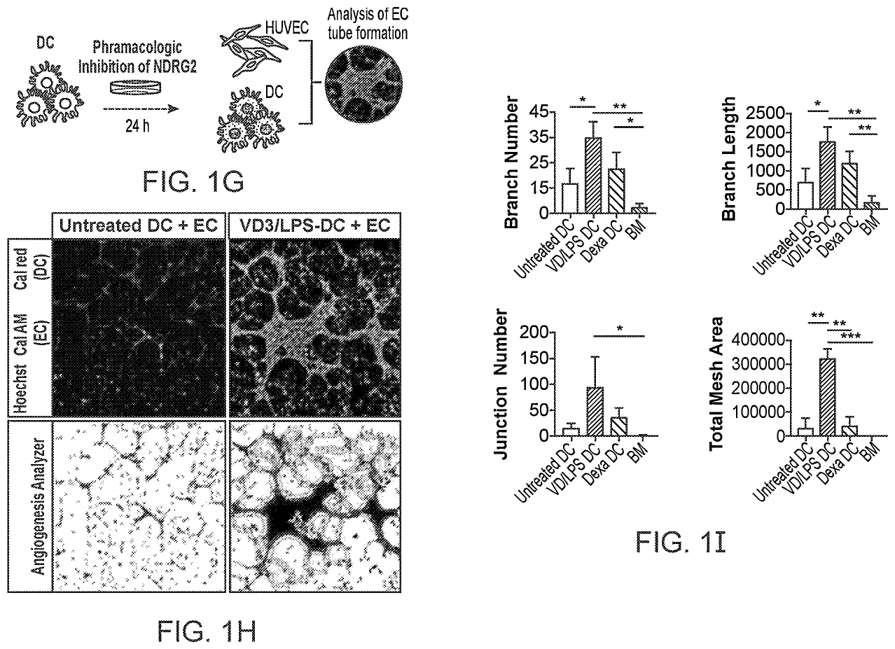

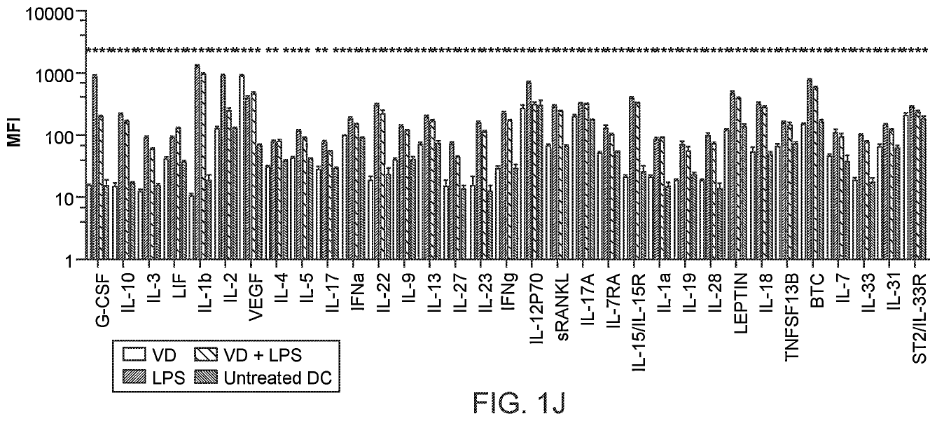

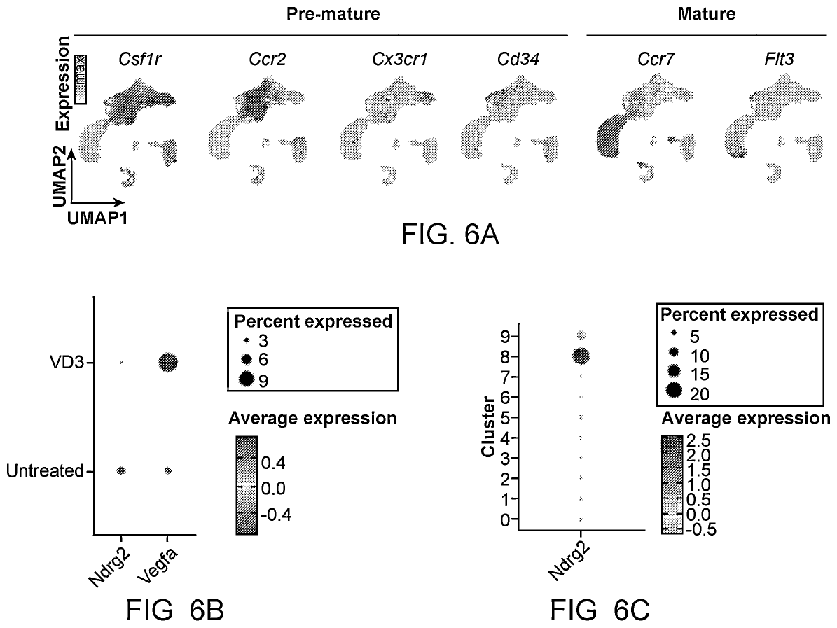

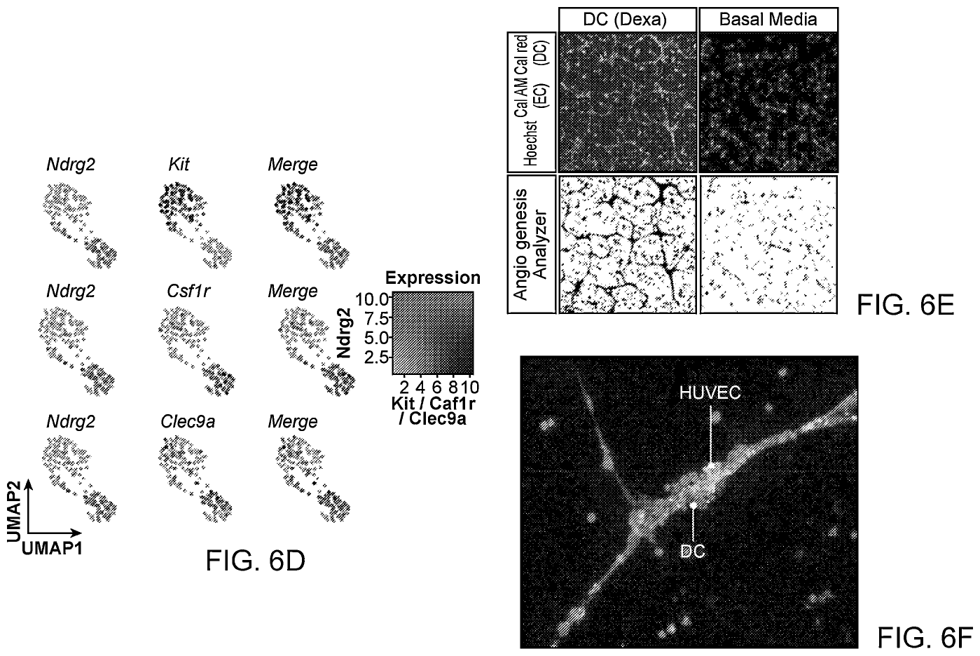

Ndrg2 expression is reduced in tolerogenic DCs. To identify potential targets for gene editing in DCs, transcriptomic profiles of tolerogenic DCs were compared, for which a variety of clinical benefits have been described, with untreated DCs. Bone marrow-derived DCs were cultivated from wild-type (WT) mice (C57/BL6) according to standard protocols. Vitamin D3 (calcitriol, VD3) was used to induce tolerogenic DCs and scRNA-seq of VD3 stimulated and untreated DCs was performed (FIG. 1a, b). Nine transcriptionally distinct clusters resembling pre-mature (characterized by high expression of Cd34, Csf1r (encoding CD115), Ccr2, and Cx3cr1) and mature BM-DCs (characterized by high expression of FIt3 (encoding CD135) and Ccr7) were identified (FIG. 1b, FIG. 6 a). Tolerogenic DCs induced by VD3 were found to show a downregulation of Ndrg2, which has previously been reported to inhibit growth factor expression, angiogenesis, and cell proliferation. In accordance, tolerogenic DCs showed an overexpression of vascular endothelial growth factor (Vegfa), indicating pro-angiogenic properties, potentially related to Ndrg2 inhibition, and making Ndrg2 an interesting target for further investigation (FIG. 1c, FIG. 6b).

Ndrg2 marks DC progenitors. Among the untreated DCs, the strongest Ndrg2 expression was found in cluster 8 (C8, FIG. 1d, FIG. 6d), which co-expressed the hematopoietic stem cell marker Cd34, and Csf1r (encoding CD115) as well as FIt3 (encoding CD135), and Clec9a (encoding DNGR-1). This expression profile is consistent with previously defined myeloid progenitor populations, namely MDP (macrophage and dendritic cell progenitor) and CDP (common dendritic cell progenitor), for which phenotypic overlap has been described. Interestingly, Ndrg2+ progenitors also expressed Itage (CD103) while being negative for Cd8 (FIG. 1e, FIG. 6d).

Compared to untreated cells, VD3 treatment almost completely abrogated the expression of Ndrg2 and its co-expressed genes Cd34, FIt3, Itgae, and Clec9a, while inducing Kit expression (FIG. 1e, FIG. 6d). This was further confirmed with flow cytometry showing that 3% of all CD34+ DC progenitors in untreated cultures co-expressed Flt3 (CD135) and CD103, while this population was reduced to 0.8% after VD3 treatment (FIG. 1f).

Ndrg2 inhibition in DCs promotes EC tube formation in co-cultures. To investigate whether inhibition of Ndrg2 could promote a pro-angiogenic behavior in DCs, BM-DCs were co-cultured with endothelial cells (ECs) and analyzed their angiogenic capacity in EC tube formation assays. Ndrg2 was inhibited according to established protocols using VD3 and lipopolysaccharide (LPS) or dexamethasone (FIG. 1g). Ndrg2-inhibited DCs were found to stimulate EC tube formation in vitro, as indicated by a higher EC branch and junction number, longer branch length, and higher total mesh area compared to EC co-cultures with untreated DCs and ECs cultured in basal media (FIG. 1i, FIG. 6e, f). The strongest angiogenic response was found in co-cultures with VD3-treated DCs (FIG. 1i, h). This was confirmed in a Luminex multiplex ELISA for 43 proteins performed on culture media of DC monocultures, showing the highest VEGFA secretion in DCs treated with VD3, which was even higher than in DCs treated with VD3 and LPS or LPS alone (FIG. 1j).

Given the strong expression of Ndrg2 in DC progenitors and the pro-angiogenic response created pharmacologic by Ndrg2 inhibition, it was hypothesized that KO of Ndrg2 would allow us to alter the transcriptional fate and development of BM-DCs, thus allowing us to generate cells that can favorably impact the wound healing process. Furthermore, precise KO of Ndrg2 avoids pleiotropic effects of VD3 and would allow for the investigation of whether the observed pro-angiogenic features of tolerogenic DCs were in fact due to loss of Ndrg2 expression.

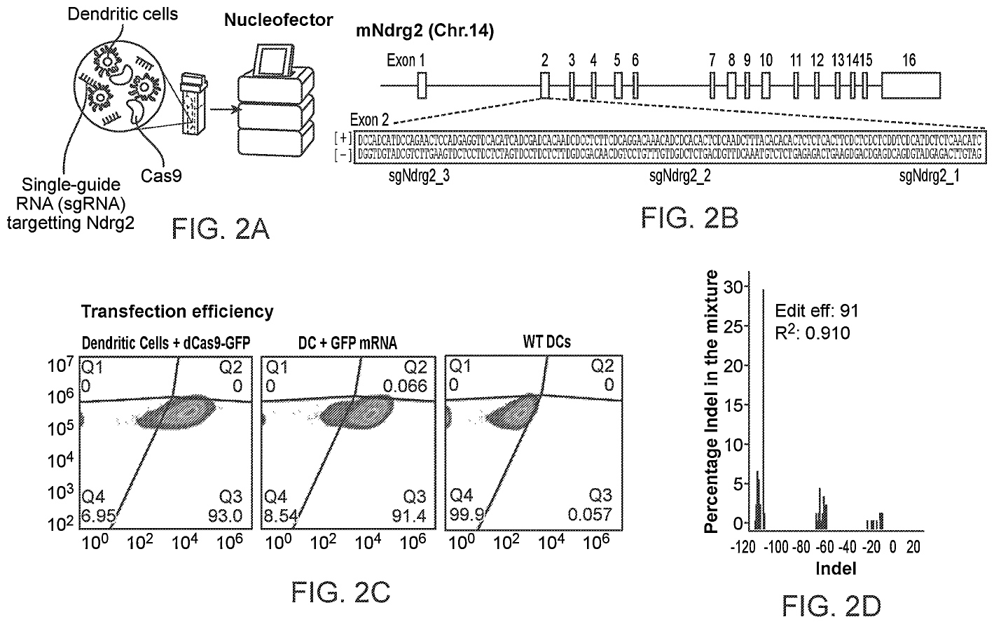

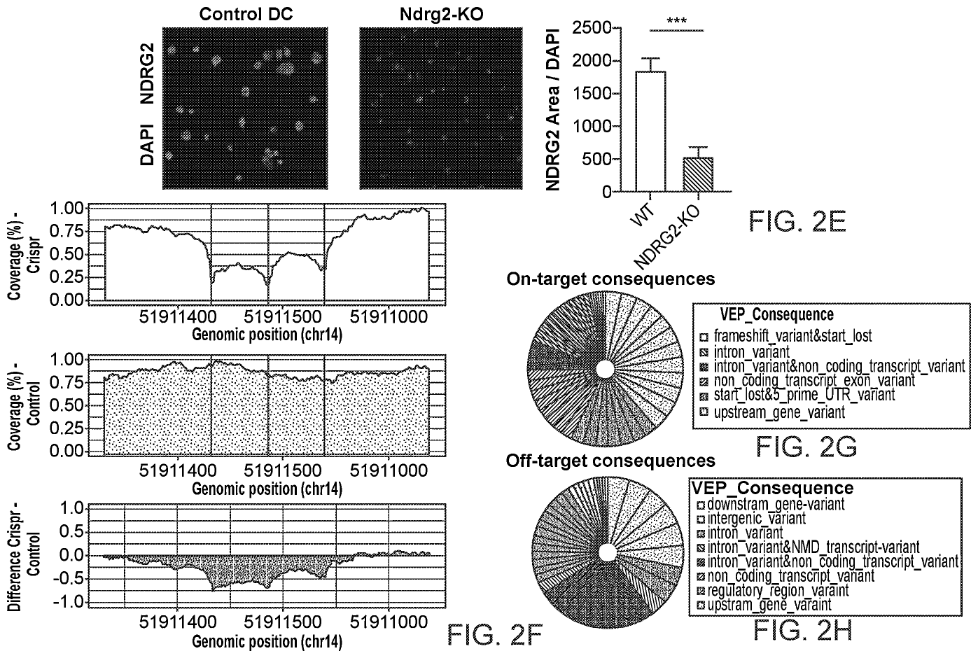

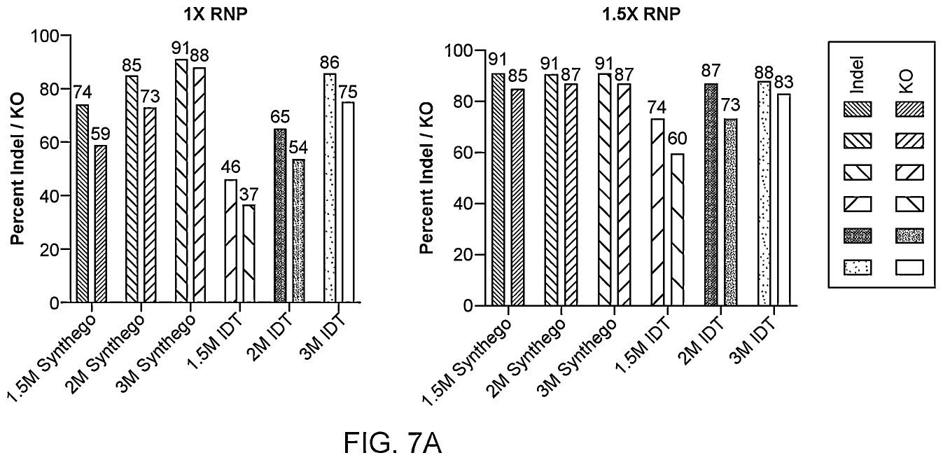

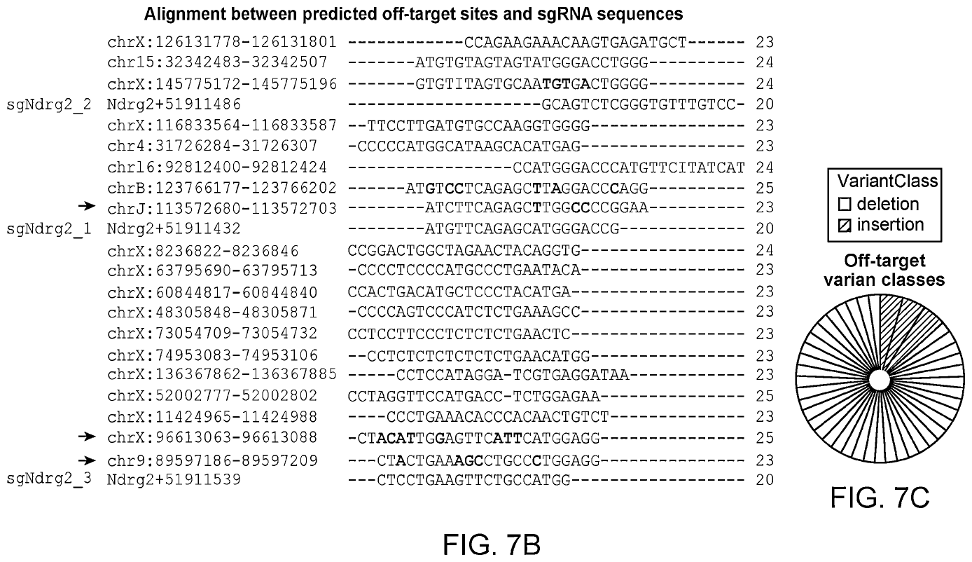

Development of a CRISPR/Cas9 platform for KO of Ndrg2 in primary DCs. To develop an approach for CRISPR/Cas9 KO of Ndrg2 in primary murine BM-DCs using nucleofection with ribonucleoprotein complexes assembled from Cas9 protein and sgRNA (Cas9-RNP; FIGS. 2a and b). This technique for CRISPR/Cas9 gene editing has recently been demonstrated to be very effective for editing of primary murine and human DCs and has several advantages compared to previously employed lentiviral vector based approaches. To achieve the highest editing efficiency, we compared indels and KO rates after Cas9-RNP nucleofection of DCs using two sets of three sgRNAs from different manufacturers targeting the Ndrg2 genomic locus as well as different numbers of cells per reaction, and different RNP concentrations (FIG. 7a, Table 1). After optimization, using and GFP-fused Cas9 protein (dCas9-GFP) and flow cytometry, a>90% transfection rate of DCs was confirmed with the approach for nucleofection (FIG. 2c). Sanger Sequencing and the Interference of CRISPR Editing (ICE) tool (Synthego) were used to compute indel and KO rates which were 91% resp. 88% using a triple-guide RNP approach (FIG. 2d, 7a, Methods). Immunocytochemical staining of CRISPR-edited and untreated DCs was performed in vitro and observed a significant reduction in Ndrg2 protein expression (FIG. 2e).

TABLE 1

| | | sgRNA targeting | SEQ ID |
|---|---|---|---|
| NDRG2 sgRNA | sgRNA Space Sequence | Exon/intron | NO: |
| NDRG2.1.AC | GTAAAAGTGACCGAGCCATA | Exon4 | 1 |
| NDRG2.1.AA | CCAGCCACTGTTTCGGTTCG | Exon5 | 2 |
| NDRG2.1.AE | ACAATAATTGGAGTTGGTGT | Exon7 | 3 |
| NDRG2_1 | ATGTTCAGAGCATGGGACCG | Exon2 | 4 |
| NDRG2_2 | GCAGTCTCGGGTGTTTGTCC | Exon2 | 5 |
| NDRG2_3 | CTCCTGAAGTTCTGCCATGG | Intron2 | 6 |
| Negative control | | | |
| sgRNA | | | |
| NC1 | CGTTAATCGCGTATAATACG | | 7 |
| NC2 | CATATTGCGCGTATAGTCGC | | 8 |
| NC3 | GGCGCGTATAGTCGCGCGTA | | 9 |
| Primer for PCR and | | | |
| sequencing | Primer sequence | | |
| NDRG2.1.AC_For | TCTGCATCCCTCCCATTTATCCTCT | | 10 |
| NDRG2.1.AC_Rev | CAGAACCCACCCTCTTGCTCTGATG | | 11 |
| NDRG2.1.AA_For | CCATGATGTAGGCCTCAACTGTAAGAC | | 12 |
| NDRG2.1.AA_Rev | TCTTCTCTGGAGCAACCTCCGCA | | 13 |
| NDRG2.1.AE_For | CAGAGAGCCGAGAATTGATAAACTC | | 14 |
| NDRG2.1.AE_Rev | GCAGGAACTCGGACCATTCCTGCAA | | 15 |
| NDRG2-3mix-F | CTGGTGTTGCTCTGCACCTTGCATTG | | 16 |
| NDRG2-3mix-R | TGGTTACCTGTCCCTGGTCCAGGAG | | 17 |

Gene editing with Cas9-RNP showed no observable off-target effects. To analyze specific mutations introduced by CRISPR gene editing in DCs at the target site as well as potential off-target effects, whole genome sequencing (WGS) with an ultrahigh depth (˜100× coverage) of CRISPR-edited DCs was performed using our triple-guide RNP approach (FIG. 2f). Wild-type DCs were analyzed as controls. A variety of different on-target effects were identified, the most common of which were upstream gene variants, followed by 5′ UTR variants (FIG. 2g). Cas-OFFinder was used to identify potential off-target sites, which allows for unbiased extensive mutation searching. Importantly, indels were not detected within 15 base pairs (bp) up- and downstream of the sites predicted by Cas-OFFinder. When each site was broadened to 200 bp up- and downstream, indels in 14 sites were identified (FIG. 2h, Table 2). By manual inspection of these loci, we found that only 7 loci showed indels that occurred only in the Ndrg2-KO group and not in the control group. After identifying the most likely off-target sites, further alignment of the sequence between the predicted Cas-OFFinder cut sites and the three sgRNA sequences targeting Ndrg2 was performed. Among the three sites that showed the highest alignment, only a protospacer adjacent motive (PAM) in two of the sites was identified. All other predicted off-target sites did not show similarity to the sgRNA sequences and therefore are very unlikely true off-target sites (FIG. 7b, c). This set of analysis together confirmed that the designed guide RNAs and the use of RNP in DCs cells had little or no observable off-target effects.

TABLE 2

| GenomicPosition | REF | ALT | AffectedGene | VEP_Consequence | VariantClass |
|---|---|---|---|---|---|
| chr3: 113572900 | ATG | A | Amy1 | intron_variant | deletion |
| chr3: 113572900 | ATG | A | Amy1 | intron_variant | deletion |
| chr3: 113572900 | ATG | A | Amy1 | intron_variant | deletion |
| chr3: 113572900 | ATG | A | Amy1 | intron_variant | deletion |
| chr4: 31726093 | T | TG | | intergenic_variant | insertion |
| chr9: 89597121 | AT | A | Minar1 | intron_variant | deletion |
| chr9: 89597121 | AT | A | Minar1 | intron_variant&NMD_transcript_variant | deletion |
| chr15: 32342480 | GA | G | Sema5a | intron_variant | deletion |
| chr15: 32342480 | GA | G | Sema5a | intron_variant&non_coding_transcript_variant | deletion |
| chr15: 32342480 | GA | G | Sema5a | intron_variant&non_coding_transcript_variant | deletion |
| chr15: 32342480 | GA | G | Sema5a | intron_variant&non_coding_transcript_variant | deletion |
| chr15: 32342480 | GA | G | Sema5a | intron_variant&non_coding_transcript_variant | deletion |
| chr15: 32342480 | GA | G | Sema5a | intron_variant&non_coding_transcript_variant | deletion |
| chr15: 32342480 | GA | G | Sema5a | intron_variant&non_coding_transcript_variant | deletion |
| chrX: 8236875 | GT | G | Ftsj1 | downstream_gene_variant | deletion |
| chrX: 8236875 | GT | G | Ftsj1 | downstream_gene_variant | deletion |
| chrX: 8236875 | GT | G | Ftsj1 | downstream_gene_variant | deletion |
| chrX: 48305938 | AT | A | Gm7212 | upstream_gene_variant | deletion |
| chrX: 48305938 | AT | A | Gm22612 | upstream_gene_variant | deletion |
| chrX: 52002943 | AACAC | A | | intergenic_variant | deletion |
| chrX: 63795577 | TCA | T | | regulatory_region_variant | deletion |
| chrX: 63795577 | TCA | T | | intergenic_variant | deletion |
| chrX: 73054780 | ATC | A | Gm6858 | downstream_gene_variant | deletion |
| chrX: 73054780 | ATC | A | | regulatory_region_variant | deletion |
| chrX: 96613198 | TTTG | T | | intergenic_variant | deletion |
| chrX: 116833769 | GTA | G | | intergenic_variant | deletion |
| chrX: 126131620 | TA | T | Gm14957 | intron_variant&non_coding_transcript_variant | deletion |
| chrX: 136367996 | G | GT | Gm14998 | downstream_gene_variant | insertion |
| chrX: 136367996 | G | GT | Gm14993 | upstream_gene_variant | insertion |

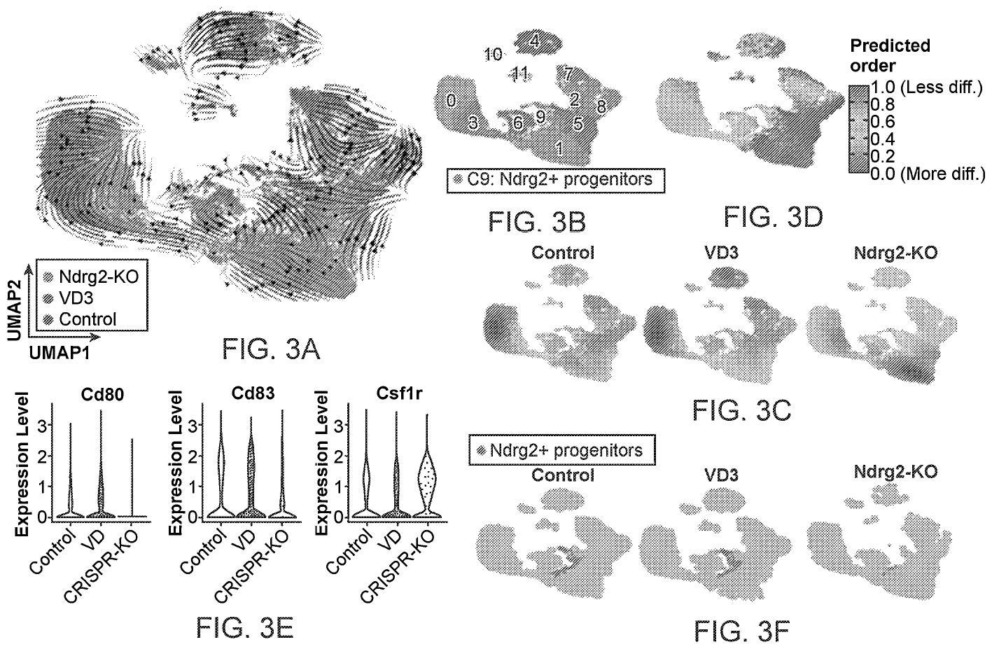

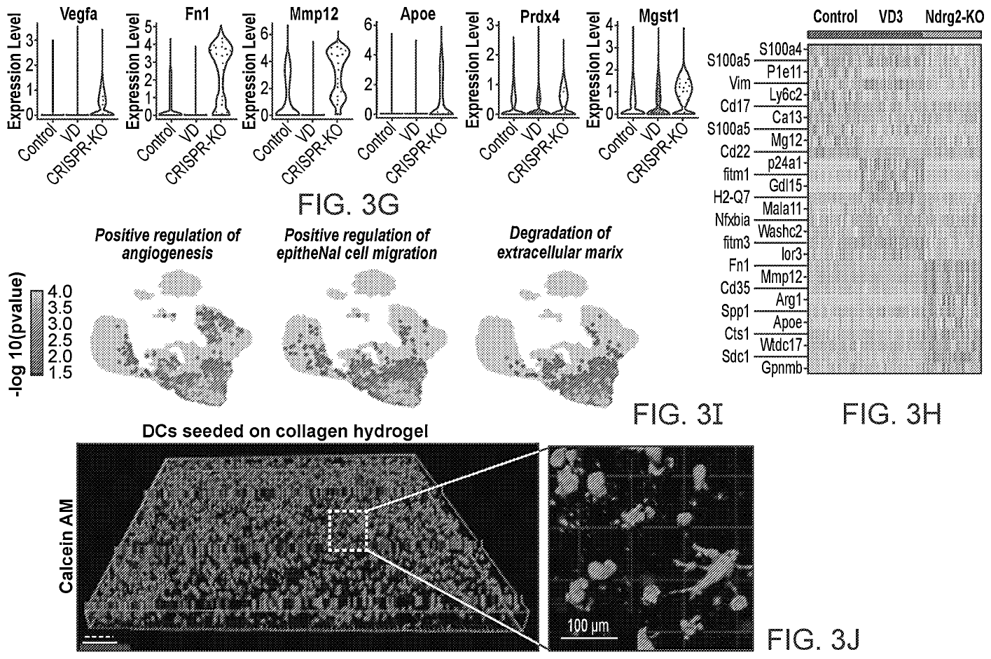

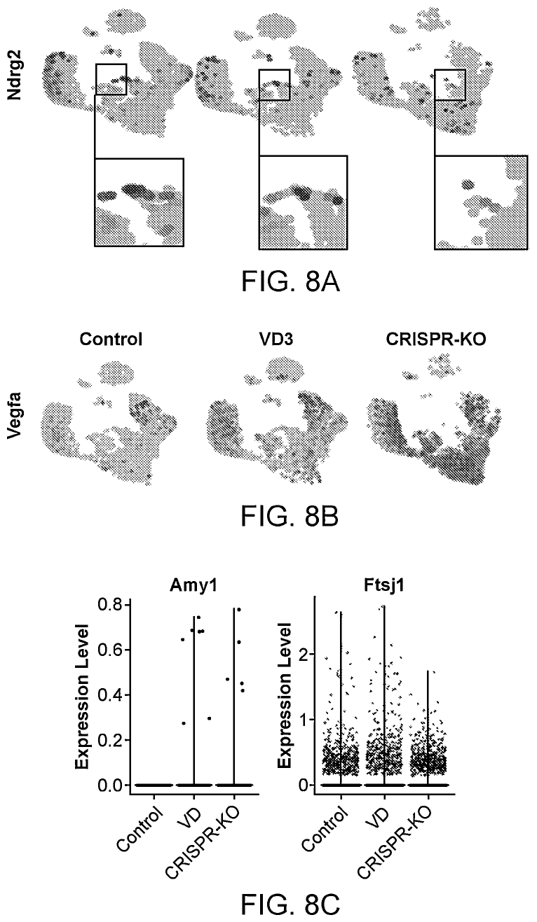

Ndrg2-KO prevents DC maturation and induces regenerative gene expression profiles. To assess the impact of Ndrg2-KO on the transcriptional fate of DCs, scRNA-seq of CRISPR-edited DCs and compared their transcriptomic profiles were performed with those of untreated DCs and DCs treated with VD3, which pharmacologically inhibits Ndrg2 (FIG. 1a). A total of 23,871 cells were analyzed (FIG. 3a). RNA velocity analysis was employed using dynamical modeling with the scVelo package to determine changes in DC differentiation in response to Ndrg2-KO. A differentiation stream was identified originating from the Ndrg2+ progenitors (cluster 9) that progressed through clusters 1 and 3 and ended in the mature DCs in cluster 0 (FIG. 3a, b). Most Ndrg2-KO cells were found in cluster 1 whereas control and VD3 treated cells mostly localized to cluster 0 (FIG. 3c). Using CytoTRACE, the relative differentiation states of individual cells based on the distribution of unique mRNA transcripts was compared and it was found that cluster 1, which mostly contained Ndrg2-KO cells, was among the least differentiated clusters, whereas cluster 0, that contained mostly VD3 treated and untreated cells, showed more advanced differentiation states (FIG. 3d). In accordance, KO of Ndrg2 led to a downregulation of the DC maturation markers Cd80 and Cd83 and an upregulation of Csf1r, which marks premature DCs (FIG. 3e). Hence, our findings indicate that Ndrg2 is highly expressed in DC progenitor cells and promotes DC differentiation toward mature phenotypes. In addition to a reduction of global Ndrg2 expression (FIG. 8a), Ndrg2-KO using our Cas9-RNP approach almost eliminated cluster 9, which contained the Ndrg2+ progenitors and thereby halted DC maturation, preserving a pre-mature DC state (FIG. 3f). These engineered pre-mature DCs exhibited an even stronger Vegfa expression than DCs treated with VD3, and in addition exhibited an up-regulation of extracellular matrix associated genes that promote wound healing, such as Fn1 (encoding fibronectin-1) and Mmp12 (matrix metalloproteinase-12) as well as anti-oxidative genes, such as Prdx4 and Mgst1 (FIG. 3g, FIG. 8b). By contrast, control DCs highly expressed pro-inflammatory markers, such as S100a6 and S100a4 (FIG. 3h).

To investigate differential regulation of cellular signaling pathways between conditions, GeneTrail 3 was used, a computational pipeline for over-representation analysis (ORA) of specific gene sets on a single-cell level, and found a significant upregulation of pathways related to angiogenesis, epithelial cell migration and degradation of the ECM in Ndrg2-KO cells, indicating a beneficial impact on wound healing (FIG. 3i).

When analyzing the expression levels of genes located at the aforementioned potential off-target sites, Amy1, Ftsj1, Sema5a, and Minar1 (Table 2), no detectable expression among all groups was found for Sema5a and Minar1. Ftsj1 and Amy1 were expressed at very low levels in less than 6% resp. 0.06% of all cells without significant differences, confirming that our approach for gene editing is highly specific and does not cause significant off-target effects (FIG. 8c).

Hydrogel delivery of Ndrg2-KO DCs promotes healing of non-diabetic wounds. Having developed and characterized the approach for gene editing to specifically induce regenerative gene expression profiles in DCs, the next goal was to test the suitability of these cells as a cell-based therapy for wound healing. The effectiveness and biocompatibility of hydrogels for cell delivery has been shown in murine and porcine wound models in combination with multiple cell types. Engineered DCs were seeded onto a previously developed pullulan-collagen hydrogel to facilitate effective cell delivery onto the wound bed. High viability and uniform distribution within the hydrogel were confirmed using calcein AM staining of live cells cultured on these hydrogels for 7 days (FIG. 3j).

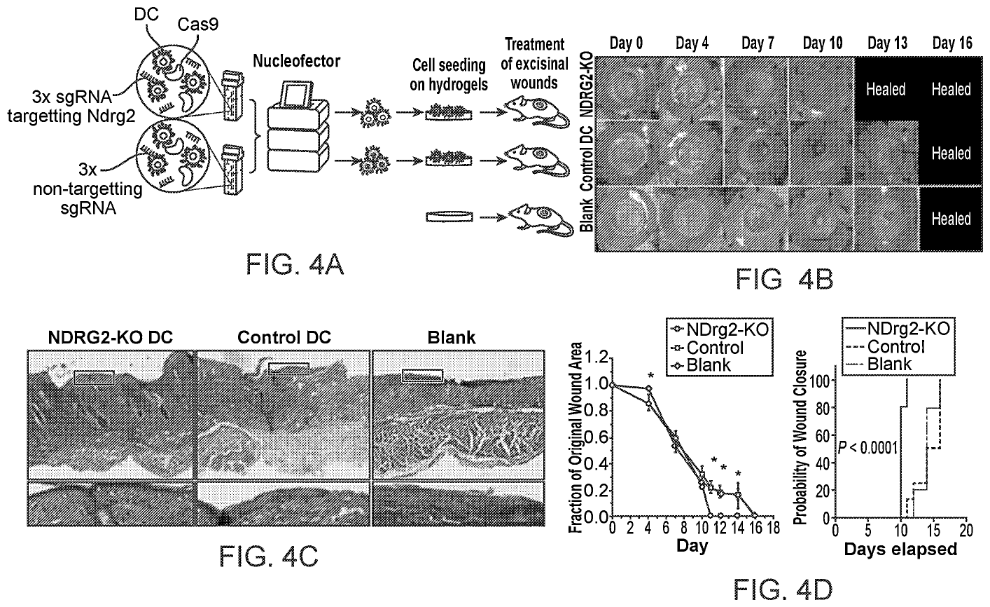

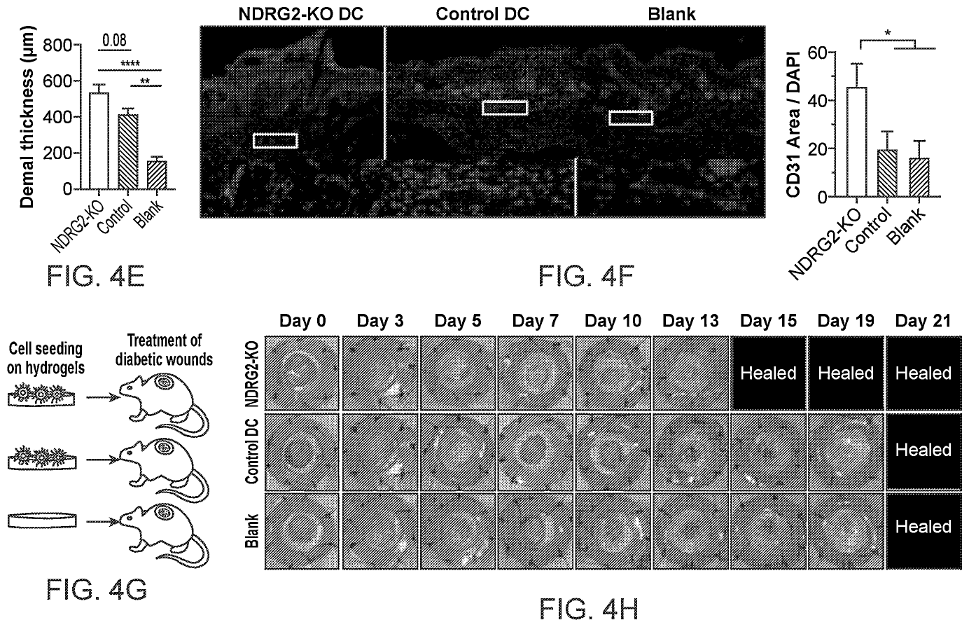

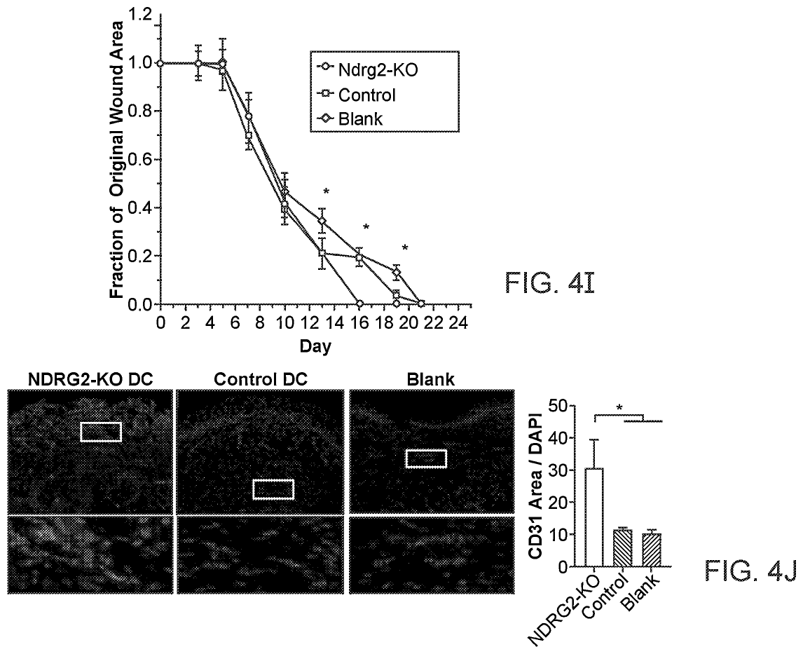

To determine the therapeutic potential of Ndrg2-KO-DCs, splinted full-thickness excisional wounds in wild-type mice (C57BL6) were treated with hydrogels seeded with either control or Ndrg2-KO DCs. For controls, DCs were subjected to the same Cas9-RNP protocol using 3 non-targeting sgRNAs, to account for any potential effect of the Cas9 RNP or the nucleofection itself on wound healing. A second control group of wounds treated with blank, unseeded hydrogels was used (FIG. 4a). Wounds treated with our engineered Ndrg2-KO DCs healed significantly faster and were completely re-epithelialized by day 11 and 5 days faster than in both control groups (FIG. 4b, c, d). Histologically, healed wounds treated with Ndrg2-KO-DCs showed a fully regenerated epithelium and a thicker dermis compared to wounds treated with control DCs or blank hydrogels (FIG. 4c-e). Immunofluorescent staining for CD31 revealed a significantly stronger vascularization of healed wound tissue that had been treated with engineered DCs compared to the control groups, confirming that the engineered DCs promoted angiogenesis and accelerated wound healing (FIG. 4f).

Hydrogel delivery Ndrg2-KO DCs promotes healing of diabetic wounds. The novel DC therapy was investigated to determine if it would also be beneficial for chronic wounds with impaired healing potential, such as in diabetes mellitus. Therefore, excisional wounds in db/db mice were treated with either Ndrg2-KO-DCs, control DCs (nucleofection with 3 non-targeting sgRNAs) or blank hydrogels (n=5 per group, FIG. 4g). These mice have a homozygous mutation in the leptin receptor gene (Lepr), resulting in morbid obesity, chronic hyperglycemia, and pancreatic beta cell atrophy. Excisional wounds in db/db mice show a significant delay in wound closure, decreased granulation tissue formation, severely impaired vascularization, and reduced cell proliferation compared to WT and other diabetic mice.

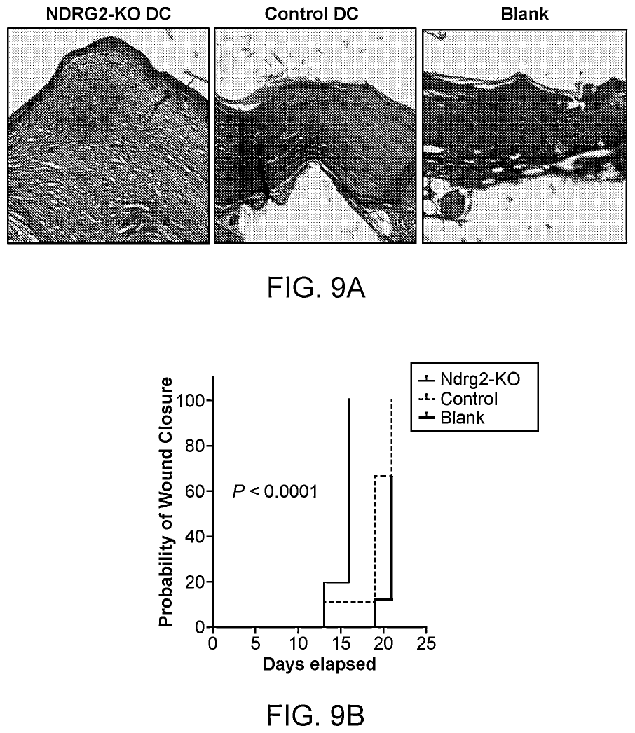

In accordance with our findings in WT mice, Ndrg2-KO DCs significantly accelerated wound healing in db/db mice, leading to complete re-epithelialization by day 16, and thus 5 days earlier than in the control groups (FIG. 4h, l, FIG. 9a,b), similar to what was observed in WT mice. Just like in WT mice, the engineered DCs also promoted increased angiogenesis, significantly improving vascularization of diabetic wound tissue compared to control DCs and unseeded hydrogels (FIG. 4j).

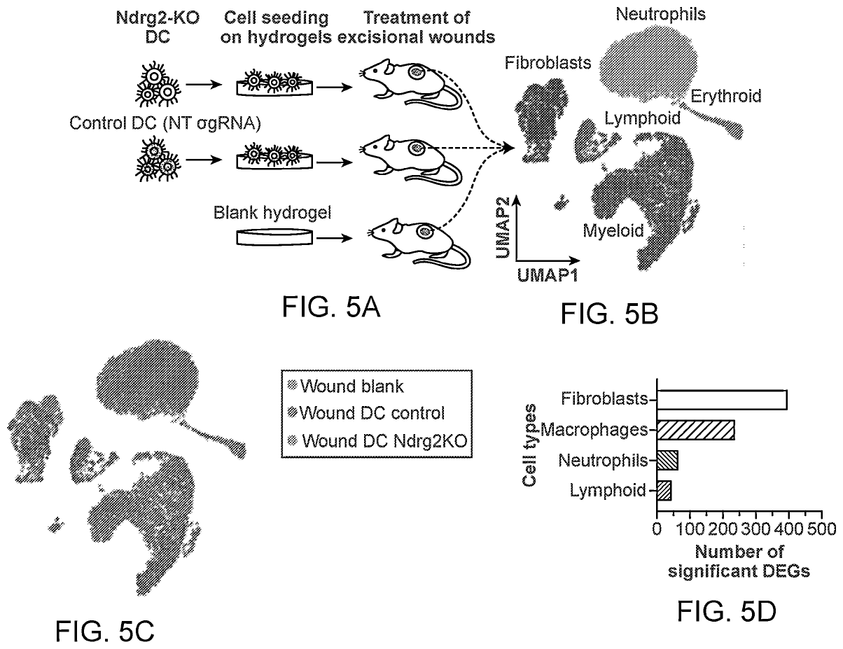

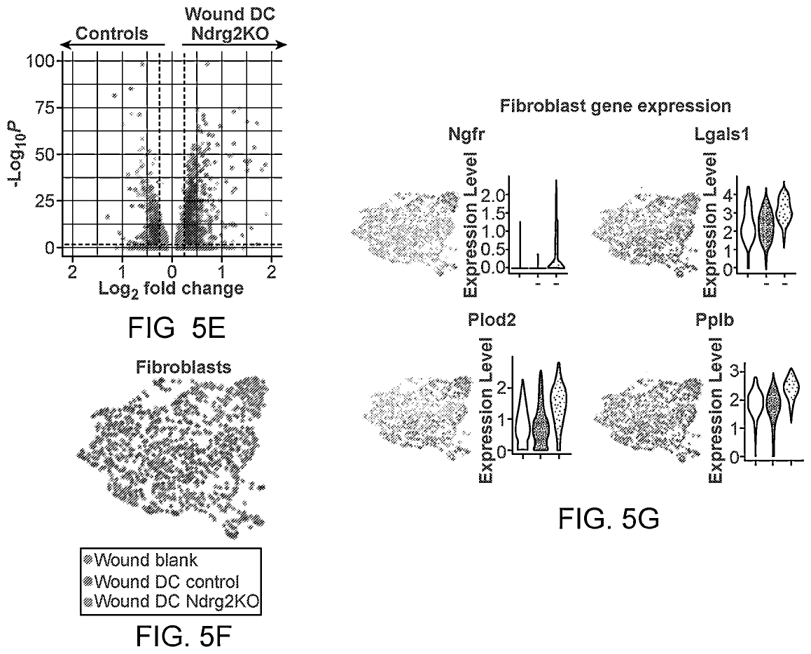

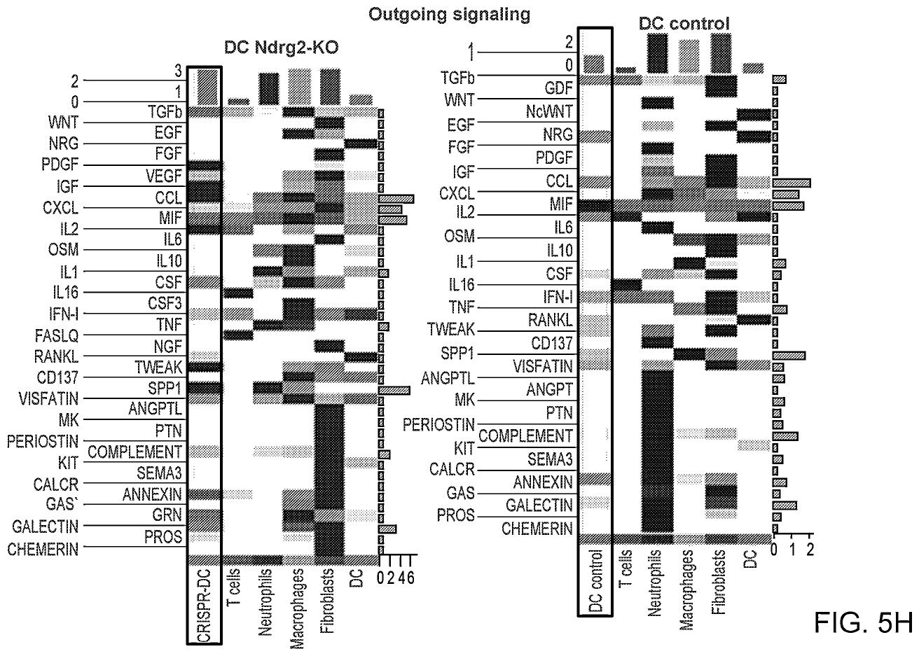

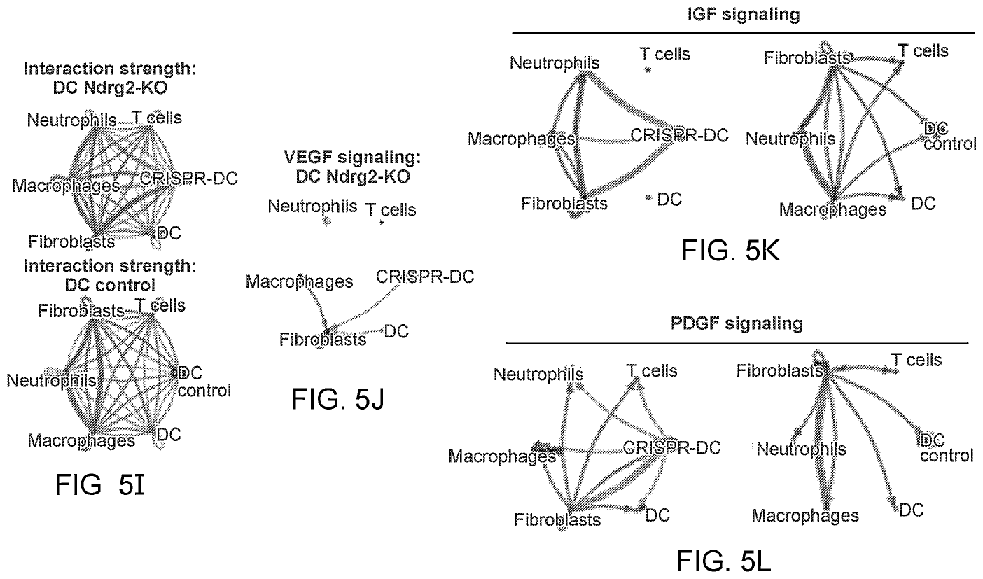

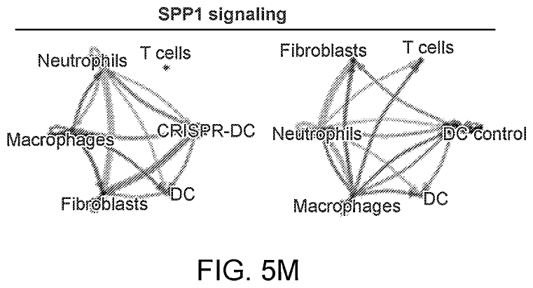

Ndrg2-KO DCs target wound fibroblasts via growth factor signaling. To interrogate how the transplanted DCs interact with different cell types of the wound healing environment and affect their individual transcriptional signatures, we performed scRNA-seq of wound tissue treated with Ndrg2-KO-DCs, control DCs, or blank hydrogels (FIG. 5a). Tissue was explanted from a second batch of mice (n=5 per group) on day 10 after wounding, when the wound healing curves of the different groups began to diverge (FIG. 4d), indicating critical differences in wound biology. scRNA-seq was performed on 10,612 cells across the three conditions, which were identified as fibroblasts, myeloid cells, neutrophils, lymphoid cells, and erythrocytes (FIG. 5b, c). Fibroblasts had the highest number of differentially expressed genes (DEGs, FC>0.5, p<0.05) between the groups among all cell types, indicating that our engineered DC therapy had the strongest impact on fibroblast gene expression within the wound bed (FIG. 5d, e). Among the most differentially expressed genes expressed by fibroblasts, several genes that have been shown to be critically involved in wound healing were found. Fibroblasts from wounds treated with Ndrg2-KO DCs almost exclusively expressed Ngfr, the receptor for nerve growth factor, which has been shown to promote cell proliferation, angiogenesis, and wound healing (FIG. 5f, g). Moreover, treatment with Ndrg2-KO DCs strongly upregulated the expression of Lgals1 (encoding galectin-1) in fibroblasts, which promotes wound healing in diabetic and non-diabetic wounds. Interestingly, an upregulation of Plod2, encoding lysyl hydroxylase 2, was found which catalyzes the first step of collagen cross linking, and Ppib, encoding peptidyl prolyl cis/trans isomerase, which catalyzes the folding of ECM proteins such as collagen. These findings indicate that wound fibroblasts may be activated by Ndrg2-KO DCs to increase ECM protein synthesis and collagen cross linking in the proliferative phase of wound healing, leading to accelerated wound closure.

To study the intercellular communication networks between the transplanted DCs and the local cells of the wound, scRNA-seq data from Ndrg2-KO and control DCs was integrated (FIG. 3a) with transcriptomic data from the respective local wound environments (FIG. 5b) and used CellChat to analyze ligand-receptor interactions.

It was found that Ndrg2-KO DCs showed a stronger outgoing signaling activity within their wound environment compared to control DCs (FIG. 5h), and also demonstrated a stronger total interaction strength within the aggregated cell-cell communication network (FIG. 5i). CellChat analysis demonstrated significant VEGF signaling from Ndrg2-KO DCs to fibroblasts, which was not present in control DCs, confirming our previous observations (FIG. 5j). In contrast to control DCs, Ndrg2-KO DCs also communicated with wound fibroblasts via other growth factor pathways, such as the insulin-like growth factor (IGF) and platelet-derived growth factor (PDGF) pathways (FIG. 5l,m), which stimulate cell proliferation and angiogenesis, and showed an upregulation of Spp1 (osteopontin) signaling, which has been shown to be a critical driver of early wound repair in diabetic wounds (FIG. 5m).

### DISCUSSION

Here, we have expanded the use of DC immunotherapy to complex diabetic and non-diabetic wounds and developed an effective gene editing approach to boost its therapeutic efficacy. While stem cells have been heavily investigated in pre-clinical studies as cell-based therapies for the treatment of wounds, the translational advancements many have hoped for have not been made over the past decades. By contrast, adoptive cell transfer using a patient's own T cells or DCs has been successfully translated to the clinic and led to oncologic breakthroughs in recent years. Combining gene editing with immunotherapy enabled the development of CAR T cell therapy, which demonstrated unsurpassed effectivity against hematologic malignancies and a potential cure for patients who are refractory to standard chemotherapies. In addition to T cells, using a patient's own DCs to treat solid tumors and multiple auto-immune conditions has demonstrated adequate safety profiles and strong therapeutic efficacy.

Gene editing of DCs for non-therapeutic use has previously been performed using lentiviral vector-based approaches. This technique, however, has several disadvantages for the development of clinical-grade DCs for cell-based therapies. Lentiviral transduction of DCs itself presents considerable challenges compared to other cell types. As professional antigen-presenting cells (APCs), DCs naturally express viral restriction factors, such as SAM domain and HD domain-containing protein 1 (SAMHD1), which allow them to deplete the cytoplasmic pool of deoxynucleoside triphosphates (dNTPs) necessary for the reverse transcription of viral genome. Hence, previous studies have reported relatively low transduction efficiencies (<40%) or required very high vector doses, which can induce undesired DC maturation or even cell toxicity. Approaches to induce proteasomal degradation of SAMHD1 using Vpx or shRNA-mediated down-regulating of SAMHD1 to improve transduction efficiency have been described; however these techniques are not suitable for the development of clinical-grade DC therapies, since they increase the risk for cell infection with viruses and induce a cytotoxic T cell response.

Recently, Cas9 RNP-based gene editing of CAR T cells have been implicated for enhanced solid tumor killing. Furthermore, despite early in the clinical test stage, Cas9 RNP editing of murine primary DCs has been demonstrated to be an effective approach leading to high KO rates for several target genes, while preserving cell viability and in vitro functional characteristics. Unlike vector-based approaches, Cas9 RNP editing does not require the cellular transcription and translation of Cas9 to generate functional Cas9-sgRNA complexes, thus enabling higher peak Cas9 expression after transfection. In addition, rapid protein degradation of Cas9 from cells has been shown to increase CRISPR specificity and reduce the exposure of cells to Cas9, thus minimizing off-target effects.

We have developed a novel approach for KO of Ndrg2 in premature DCs using nucleofection with triple-guide Cas9-RNPs which allows for streamlined, robust and high-throughput editing of large quantities of DCs without the need for viral vectors. Our platform for gene editing consistently achieved >90% KO rates in primary DCs. This study is the first to comprehensively assess off-target effects after Cas9 RNP nucleofection of DCs via ultra-deep sequencing, demonstrated a high specificity of our CRISPR without detectable off-target effects.

Previous studies reported that Ndrg2 is involved in DC maturation, however, its exact role has been poorly defined so far. Using scRNA-seq of over 20,000 BM-DCs, we show that Ndrg2 marks MDP/CDPs and that its knockout prevents DC maturation, preserving an immature cell state that is characterized by strong regenerative and pro-angiogenic gene expression profiles that cannot be achieved by pharmacologic treatment with VD3. In addition, targeted alteration of cells by CRISPR/Cas9 gene editing has a higher specificity and avoids pleiotropic effects of pharmacologic cell treatment. Moreover, CRISPR-KO provides a durable effect on cells, since target genes are eliminated, which may be of particular importance for future translational applications when genetically edited cell products are stored, or clinical application is delayed.

DCs edited with this approach were suitable for seeding on delivery hydrogels for in vivo application as a cell-based therapy. The collagen-pullulan hydrogels used in this study provides an optimal environment similar to native ECM and have been demonstrated to be an ideal vehicle for efficient cell delivery. In addition, these hydrogels, even without cells, have a positive impact on wound healing. Wounds treated with control DCs did not heal faster compared to those treated with unseeded hydrogels, which indicates that DCs alone may not provide an additional benefit over collagen-pullulan hydrogels for wound healing. However, KO of Ndrg2 induced regenerative transcriptomic profiles in DCs, which led to significantly accelerated wound healing of WT as well as diabetic wounds by 5 days, surpassing what has previously been reported for genetically edited MSCs.

When analyzing the cellular ecology of the healing wounds in the proliferative phase of wound healing at day 10, we found that our edited DCs specifically direct growth factor signaling (VEGF, PDGF and IGF) toward fibroblasts of the wound bed. Fibroblasts are among the most critical cell types in wound healing and showed an upregulation of wound healing and ECM related genes after treatment with edited DCs, which indicates their strong therapeutic response and provides a molecular basis for the observed accelerated wound closure with edited DCs.

In summary, our genetically edited DC therapy provides a highly effective, robust, and easy-to-use approach for the treatment of chronic wounds. It provides strong evidence and allows DC therapy combined with gene editing to provide significant translational opportunities to help patients with diabetic and non-diabetic ulcers.

### Methods

Isolation and cultivation of BM-DCs. Cultivation of DCs from the murine bone marrow was performed according to previously published protocols. Briefly, after euthanasia, femurs and tibias were excised and the epiphyses on both ends of the long bones were removed. Bone marrow was collected by flushing the bones with 5 ml RPMI. Red blood cells were lysed with ACK lysis buffer. Cells were strained and then washed in complete media. Cells were cultured at a concentration of 2×10⁶ per 100 mm culture dish in 10 ml RPMI containing 10% fetal bovine serum (FBS), 100 U/ml penicillin, 0.1 mg/ml streptomycin, 2 mM glutamine, 50 μM 2-mercaptoethanol, and 200 murine granulocyte-macrophage colony-stimulating factor (GM-CSF, Peprotech, Frankfurt, Germany). On day 3, 10 ml complete media were added to the culture dishes and on day 6, 10 ml of media were collected, cells were spun down and re-suspended in fresh media. For all experiments, only cells in suspension that represent the DC fraction of the cultures were used, and adherent cells were discarded. For in vitro experiments, DCs were treated on day 7 of culture for 24 h with either 10⁻⁶M 1,25 dihydroxy-cholecalciferol (Sigma-Aldrich, St. Louis, MO), 10⁻⁶M 1,25 dihydroxy-cholecalciferol and 100 ng/ml LPS (Escherichia coli, 026:B6; ThermoFisher) or 10⁻⁷ M dexamethasone (Sigma Aldrich).

Endothelial cell tube formation assay. Human umbilical vein endothelial cells (HUVECs) were obtained from Cell Applications (San Diego, CA). Tube formation assays were performed according to previously published protocols. Briefly, HUVECs were seeded at 2×10⁵ cells per 75 cm² in T75 culture flasks and expanded in Human EC Growth Medium (Cell Applications) until 75% confluence was reached. After washing off growth media, cells were serum starved one day before the assay by incubation in Human EC Basal Medium (Cell Applications) for 24 h. Calcein AM (green; ThermoFisher) was added to cultures at a concentration of 2 μg/ml to stain cells and assess cell viability. Cells were incubated with Calcein AM in the dark at 37° C. and 5% CO₂ for 30 min, then the stain was washed off using PBS. Assays were performed in 24-well plates coated with Geltrex LDEV (lactose dehydrogenase elevating virus)-Free Reduced Growth Factor Basement Membrane Matrix (ThermoFisher) at 75 μl/cm² according to the manufacturer's recommendations. Cells were plated at a concentration of 3.5×10⁴ in 200 μl Human EC Basal Medium (Cell Applications) per well. For co-culture assays, DCs were stained using CellTrace Calcein Red-Orange (ThermoFisher) and added to the EC cultures at a 1:1 ratio. Assays were run for 12 h and then fixed using 4% paraformaldehyde and imaged using a Zeiss LSM 880 confocal laser scanning microscope.

CRISPR/Cas9-mediated Ndrg2 knockout and efficiency analysis. Single-guide RNA (sgRNA) design was performed using design tools from Integrated DNA Technologies (IDT, Newark, NJ) and Synthego (Menlo Park, CA). All spacer sequences of Ndrg2 sgRNA, positive and negative control sgRNAs, primers for PCR amplification and DNA Sanger sequencing are listed in table 1. RNPs were assembled using Cas9 Nuclease V3 purchased from IDT and directly synthesized together with Alt-R CRISPR-Cas9 sgRNAs from IDT or three guides as part of a multi-guide design from Synthego. For nucleofection the 4D-Nucleofector System and P3 Primary Cell 4D Nucleofector X Kit L (Lonza, Basel, Switzerland). The RNP electroporation was optimized for DCs by using multiple-guide mixtures and different number of cells in a 100 uL total volume. The electroporated cells were plated in Costar Ultra-Low Attachmentat 6-well plates (Corning) at 0.5 million cells per well in 2 mL medium. The cells from one well were harvested after 48 hours and the total genomic DNA was extracted using Qiagen DNeasy Blood & Tissue kit. To analyze and verify KO efficiency, about 350 bp genome region covering 175 bp upstream and downstream of sgRNA targeting sites were amplified. The PCR products were column purified and then sequenced by Sanger Sequencing. The cutting and knockout efficiency were estimated using the ICE analysis tool (Synthego).

After several runs of optimization, we used Cas9-RNP and the Lonza 4D-Nucleofector System together with the P4 Primary Cell 4D-Nucleofector X Kit L (Lonza) to transfect and create Ndrg2-KO DCs. To generate RNP, we used 18.6 pmol of Cas9 and 22 pmol of equimolar mixture of sgNDRG2_1, sgNDRG2_2 and sgNDRG2_3 (Synthego, Table 1, FIG. 7a) per 0.5 million cells that were washed with DPBS and suspended in R Buffer (Lonza). After optimization, we processed 3 million DCs in each 100 μL electroporation system for in vitro and in vivo experiments. All other steps followed IDT (Newark, NJ) Alt-CRISPR-Cas9 protocol. Cells were transfected using the program DK-100. We used a mixture of non-targeting sgRNAs (sgNC1, sgNC2, and sgNC3, Table 1) in the control RNP electroporation. For each cell production for in vivo experiments, we processed 30 million of each Ndrg2-KO DCs and non-targeting control DCs.

Whole genome sequencing. Genomic DNA from Ndrg2-KO cells and control electroporated cells was extracted and whole genome sequencing (WGS) at ultrahigh depth (100×) was preformed to confirm KO efficacy and evaluate off-target effects. WGS was performed by Novogene Corporation Inc. (Scaramento, CA). The genomic DNA was randomly fragmented by sonication to the size of 350 bp, then DNA fragments were end polished, A-tailed, and ligated with the full-length adapters of IIIumina sequencing, and followed by further PCR amplification. The PCR products as the final construction of the libraries were purified with AMPure XP system. Then libraries were checked for size distribution by Agilent 2100 Bioanalyzer (Agilent Technologies, Santa Clara, CA), and quantified by real-time PCR (to meet the criteria of 3 nM). Samples were sequenced using IIIumina Novaseq 6000 on an S4 Flowcell. Sequencing was performed according to the manufacturer's recommendations.

Off-target analysis. The resulting reads for both samples were then aligned to the mouse reference genome (GRCm38) using Burrows-Wheeler Aligner (BWA, Version 0.7.15) in the ‘bwa mem’ setting with default parameters. To identify potential off-target sites, we followed an approach of previous studies using CRISPR-edited cells for clinical applications in human patients. First, variant calling was conducted using the strelka2 ‘somatic workflow’ setting for paired samples (Version 2.9.2). All variants were then annotated using the Ensembl Variant Effect Predictor (Version 101, assembly=“GRCm38.p6”, dbSNP=“150”, gencode=“GENCODE M25”). Additionally, the Cas-OFFinder webtool (Version 2.4) was used to predict potential off-target sites using all three sgRNA sequences. Here, sequences with five or fewer mismatches and DNA and RNA bulge sizes of two or less were searched for. To identify variants that might be potential off-target sites, only insertion or deletion events that were not detected in the control sample, that passed all quality control checks (‘FILTER=PASS’), and that did not contain the ‘SNVHPOL’ information in their VCF ‘INFO’ field were considered. Additionally, the remaining candidate sites were checked to see if they were located within a 200 bp window up- or downstream of any predicted off-target site.

Immunofluorescent staining and confocal laser scanning microscopy. After fixation, tissue was dehydrated in 30% sucrose in PBS for at least 48 hours at 4° C. Tissue was incubated in optimal cutting temperature compound (O.C.T., TissueTek, Sakura Finetek, Torrance, CA) for 24 hours at 4° C. and cryoembedded in tissue molds on dry ice. Frozen sections were performed at 7 μm thickness on a cryostat. Antigen retrieval was performed using 0.01 M sodium citrate buffer in PBS (Abcam, Cambridge, MA), followed by blocking for 2 hours in 5% goat serum (Invitrogen, Waltham, MA) in PBS. Sections were then incubated in anti-CD31 antibody (ab28364; Abcam, Waltham, MA) over night at 4° C. Secondary antibodies were applied for 1 hour at room temperature (Goat anti-Rabbit IgG (H+L) Highly Cross-Adsorbed Secondary Antibody, Alexa Fluor Plus 647, Thermo Fisher).). For staining of cultured DCs, cells in suspension were collected from the culture dishes, spun down and washed in PBS. Permeabilization was performed using 0.1% Triton X-100 in 1×PBS and cells were incubated at room temperature for 15 min. Cells were again washed and spun down, and blocking was performed using 1% BSA in 1×PBS for 1 h. Cells were incubated in recombinant Anti-NDRG2 antibody (ab174850; Abcam) in 500 μL of 0.1% BSA for 3 h, then washed three times in 1×PBS. Goat anti-Rabbit IgG (H+L) Highly Cross-Adsorbed Secondary Antibody (Alexa Fluor Plus 647, Thermo Fisher) diluted in 500 μL of 0.1% BSA was applied for 45 min. Cells were again washed three times in 1×PBS, then mounted on slides using cover slips. Imaging was performed on a Zeiss LSM 880 confocal laser scanning microscope at the Cell Science and Imaging Facility at Stanford University. To obtain high resolution images, multiple serial images of 25× magnification were acquired using automatic tile scanning. Individual images were stitched together during acquisition using the ZEN Black software (Zeiss, Oberkochen, Germany).

Quantification of immunofluorescent staining. Immunofluorescent staining was quantified using a code written in MATLAB adapted from previous image analysis studies. Briefly, confocal images were separated into their RGB channels and converted to binary to determine the area covered by each color channel. DAPI stain, corresponding to the blue channel, was converted to binary using imbinarize and an image-specific, automated threshold determined from the function graythresh in order to optimize the number of DAPI cells counted within the image. For the red and green channels, which corresponded to the actual protein stains, the images were converted to binary with a set a consistent threshold of ˜0.3 for all images of the same stain. This consistent threshold insured that our automated quantification of stain area would be unbiased. The area of red and green stains was then normalized by dividing by the number of cells, which was calculated as the number of DAPI nuclei above size threshold of 15 pixels. 3D reconstruction of confocal microscopy images was performed using Imaris 7.2 (Oxford Instruments, Abingdon, UK).

Flow cytometry Cells were washed in FACS buffer and stained with PE anti-mouse CD135 antibody, Brilliant Violet 421 anti-mouse CD103 antibody, and Alexa Fluor®647 anti-mouse CD34 antibody (Biolegend, San Diego, CA). Flow cytometry was performed on a BD FACS Aria (Becton Dickinson, San Jose, CA) and data was analyzed using FlowJo (Becton Dickinson, San Jose, CA).

Single cell RNA-seq data processing, normalization, and cell cluster identification. Base calls were converted to reads using the Cell Ranger (10× Genomics, Pleasanton, CA, USA; version 3.1) implementation mkfastq and then aligned against the mm10 (mouse) genome using Cell Ranger's count function with SC3Pv3 chemistry and 5,000 expected cells per sample. Cell barcodes representative of quality cells were delineated from barcodes of apoptotic cells or background RNA based on a threshold of having at least 300 unique transcripts profiled, less than 100,000 total transcripts, and less than 10% of their transcriptome of mitochondrial origin. Unique molecular identifiers (UMIs) from each cell barcode were retained for all downstream analysis. Raw UMI counts were normalized with a scale factor of 10,000 UMIs per cell and subsequently natural log transformed with a pseudocount of 1 using the R package Seurat (version 3.1.1). Aggregated data were then evaluated using uniform manifold approximation and projection (UMAP) analysis over the first 15 principal components. Cell annotations were ascribed using the SingleR package (version 3.11) against the ImmGen database and confirmed by expression analysis of specific cell type markers. Louvain clustering was performed using a resolution of 0.5 and 15 nearest neighbors.

Generation of subpopulation markers, over-representation analysis, and cell-cell communication analysis. Cell-type markers were generated using Seurat's native FindMarkers function with a log fold change threshold of 0.25 using the ROC to assign predictive power to each gene. Using GeneTrail 3 an ORA was performed for each cell using the 500 most expressed protein coding genes on the gene sets of the Gene Ontology. P-values were adjusted using the Benjamini-Hochberg procedure and gene sets were required to have between 2 and 1000 genes. To predict intercellular communication between transplanted DCs and local wound cells, scRNA-seq data from Ndrg2-KO DCs and control DCs (FIG. 3a) were integrated with data from their specific wound environments (FIG. 5a) using Seurat. The CellChat package was used to analyze cellular communication networks using the Secreted signaling” database.

Statistical Analysis. Data are shown as mean±SEM and were analyzed using Prism 8 (GraphPad, La Jolla, CA). Two-group comparisons were performed with Student's t-test (unpaired and two-tailed). One- or two-way ANOVA (analysis of variance), followed by Benajmini Hochberg post hoc test were performed for comparisons of >2 groups as appropriate. P<0.05 was considered statistically significant. The statistical methods used for scRNA-seq analysis are described in the specific sections above.

Animals. All experiments were performed in accordance with Stanford University Institutional Animal Care and Use Committees and the NIH Guide for the Care and Use of Laboratory Animals. The study was approved by the Administrative Panel on Laboratory Animal Care (APLAC) at Stanford University (APLAC protocol number: APB-2965-GG0619). C57BL/6J (wild-type) and C57/BL/6-db/db mice were obtained from the Jackson Laboratory (Bar Harbor, ME). All animal surgeries were performed under inhalation anesthesia with isoflurane (Henry Schein Animal Health) at a concentration of 1-2% in oxygen at 3 L/min. The mice were placed in prone position, and dorsal fur was shaved. Skin was disinfected with betadine solution followed by 70% ethanol three times.

Cell seeding on hydrogels. Cells in suspension were collected from cultures and seeded on pullulan-collagen hydrogels with a diameter of 8 mm at a concentration of 500,000 cells per hydrogel and wound, using a previously described capillary force seeding technique. We have previously demonstrated that this approach preserves cell viability as well as hydrogel architecture and enables efficient cell delivery.

Splinted excisional wound model. Splinted full-thickness excisional wounds were created as previously described by Galiano et al. Here, a silicone ring is sutured around the wound margin to stent the skin, mimicking human physiological wound healing by limiting the contraction of the murine panniculus carnosus muscle and allowing the wounds to heal by granulation tissue formation and re-epithelialization. Two full-thickness dermal wounds of 6 mm diameter were created on the dorsum of each mouse using biopsy punches. A silicone ring was fixed to the dorsal skin using an adhesive glue (Vetbond, 3M, Saint Paul, MN) as well as 8 interrupted 6-0 nylon sutures placed around the outer edge of the ring to prevent wound contraction. Collagen pullulan hydrogels seeded with DCs or blank hydrogels. were placed onto the wound bed and the wounds were covered with sterile dressings (Tegaderm, 3M, Saint Paul, MN). Digital photographs were taken immediately after wounding and placement of the splints and during every dressing change until the time of wound closure. For the hydrogel treatment study, wounds were harvested on day 16 post-wounding, when the wounds in all groups had healed.

### REFERENCES

Sen, C. K. et al. Wound Repair Regen 17, 763-771, (2009).
Nussbaum, S. R. et al. Value Health 21, 27-32, (2018).
desJardins-Park, H. E., Gurtner, G. C., Wan, D. C. & Longaker, M. T. Adv Wound Care (New Rochelle), (2021).
Veith, A. P., Henderson, K., Spencer, A., Sligar, A. D. & Baker, A. B. Adv Drug Deliv Rev 146, 97-125, (2019).
Sivaraj, D. et al. Front Bioeng Biotechnol 9, 660145, (2021).
Mushahary, D., Spittler, A., Kasper, C., Weber, V. & Charwat, V. Cytometry A 93, 19-31, (2018).
Lipsitz, Y. Y. et al. Cytotherapy 19, 1383-1391, (2017).
Banchereau, J. & Steinman, R. M. Nature 392, 245-252, (1998).
Hawiger, D. et al. Dendritic cells induce peripheral T cell unresponsiveness under steady state conditions in vivo. J Exp Med 194, 769-779, doi:10.1084/jem.194.6.769 (2001).
Thurner, B. et al. Generation of large numbers of fully mature and stable dendritic cells from leukapheresis products for clinical application. J Immunol Methods 223, 1-15, (1999).
Giannoukakis, N., Phillips, B., Finegold, D., Harnaha, J. & Trucco, M. Phase I (safety) study of autologous tolerogenic dendritic cells in type 1 diabetic patients. Diabetes Care 34, 2026-2032, doi:10.2337/dc11-0472 (2011).
Benham, H. et al. Citrullinated peptide dendritic cell immunotherapy in HLA risk genotype-positive rheumatoid arthritis patients. Sci Transl Med 7, 290ra287, doi:10.1126/scitranslmed.aaa9301 (2015).
Bell, G. M. et al. Autologous tolerogenic dendritic cells for rheumatoid and inflammatory arthritis. Ann Rheum Dis 76, 227-234, doi:10.1136/annrheumdis-2015-208456 (2017).
Zubizarreta, I. et al. Immune tolerance in multiple sclerosis and neuromyelitis optica with peptide-loaded tolerogenic dendritic cells in a phase 1b trial. Proc Natl Acad Sci USA 116, 8463-8470, doi:10.1073/pnas.1820039116 (2019).
Willekens, B. et al. Tolerogenic dendritic cell-based treatment for multiple sclerosis (MS): a harmonised study protocol for two phase I clinical trials comparing intradermal and intranodal cell administration. BMJ Open 9, e030309, doi:10.1136/bmjopen-2019-030309 (2019).
Sawitzki, B. et al. Regulatory cell therapy in kidney transplantation (The ONE Study): a harmonised design and analysis of seven non-randomised, single-arm, phase 1/2A trials. Lancet 395, 1627-1639, doi:10.1016/S0140-6736(20)30167-7 (2020).
Jauregui-Amezaga, A. et al. Intraperitoneal Administration of Autologous Tolerogenic Dendritic Cells for Refractory Crohn's Disease: A Phase I Study. J Crohns Colitis 9, 1071-1078, doi:10.1093/ecco-jcc/jjvl44 (2015).
Cheever, M. A. & Higano, C. S. PROVENGE (Sipuleucel-T) in prostate cancer: the first FDA-approved therapeutic cancer vaccine. Clin Cancer Res 17, 3520-3526, doi:10.1158/1078-0432.CCR-10-3126 (2011).
Rathi, N., McFarland, T. R., Nussenzveig, R., Agarwal, N. & Swami, U. Evolving Role of Immunotherapy in Metastatic Castration Refractory Prostate Cancer. Drugs 81, 191-206, doi:10.1007/s40265-020-01456-z (2021).
Razeghian, E. et al. A deep insight into CRISPR/Cas9 application in CAR-T cell-based tumor immunotherapies. Stem Cell Res Ther 12, 428, doi:10.1186/s13287-021-02510-7 (2021).
Hou, A. J., Chen, L. C. & Chen, Y. Y. Navigating CAR-T cells through the solid-tumour microenvironment. Nat Rev Drug Discov 20, 531-550, doi:10.1038/s41573-021-00189-2 (2021).
Stadtmauer, E. A. et al. CRISPR-engineered T cells in patients with refractory cancer. Science 367, doi:10.1126/science.aba7365 (2020).
Lu, Y. et al. Safety and feasibility of CRISPR-edited T cells in patients with refractory non-small-cell lung cancer. Nat Med 26, 732-740, doi:10.1038/s41591-020-0840-5 (2020).
Srifa, W. et al. Cas9-AAV6-engineered human mesenchymal stromal cells improved cutaneous wound healing in diabetic mice. Nat Commun 11, 2470, doi:10.1038/s41467-020-16065-3 (2020).
Nakano, H., Lyons-Cohen, M. R., Whitehead, G. S., Nakano, K. & Cook, D. N. Distinct functions of CXCR4, CCR2, and CX3CR1 direct dendritic cell precursors from the bone marrow to the lung. J Leukoc Biol 101, 1143-1153, doi:10.1189/jlb.1A0616-285R (2017).
Harada, S. et al. Flt3 ligand promotes myeloid dendritic cell differentiation of human hematopoietic progenitor cells: possible application for cancer immunotherapy. Int J Oncol30, 1461-1468 (2007).
Dieu, M. C. et al. Selective recruitment of immature and mature dendritic cells by distinct chemokines expressed in different anatomic sites. J Exp Med 188, 373-386, doi:10.1084/jem.188.2.373 (1998).
Lutz, M. B., Inaba, K., Schuler, G. & Romani, N. Still Alive and Kicking: In-Vitro-Generated GM-CSF Dendritic Cells!Immunity44, 1-2, doi:10.1016/j.immuni.2015.12.013 (2016).
Choi, S. C. et al. Expression and regulation of NDRG2 (N-myc downstream regulated gene 2) during the differentiation of dendritic cells. FEBS Lett 553, 413-418, doi:10.1016/s0014-5793(03)01030-5 (2003).
Riboldi, E. et al. Cutting edge: proangiogenic properties of alternatively activated dendritic cells. J Immunol 175, 2788-2792, doi:10.4049/jimmunol.175.5.2788 (2005).
Henn, D. et al. MicroRNA-regulated pathways of flow-stimulated angiogenesis and vascular remodeling in vivo. J Transl Med 17, 22, doi:10.1186/s12967-019-1767-9 (2019).
Liu, S. et al. NDRG2 induced by oxidized LDL in macrophages antagonizes growth factor productions via selectively inhibiting ERK activation. Biochim Biophys Acta 1801, 106-113, doi:10.1016/j.bbalip.2009.09.022 (2010).
Hu, W. et al. Emerging role of N-myc downstream-regulated gene 2 (NDRG2) in cancer. Oncotarget 7, 209-223, (2016).
Waskow, C. et al. The receptor tyrosine kinase Flt3 is required for dendritic cell development in peripheral lymphoid tissues. Nat Immunol 9, 676-683, (2008).
Poltorak, M. P. & Schraml, B. U. Fate mapping of dendritic cells. Front Immunol 6, 199, (2015).
Cabeza-Cabrerizo, M., Cardoso, A., Minutti, C. M., Pereira da Costa, M. & Reis, E. S. C. Dendritic Cells Revisited. Annu Rev Immunol 39, 131-166, (2021).
Hiatt, J. et al. Efficient generation of isogenic primary human myeloid cells using CRISPR-Cas9 ribonucleoproteins. Cell Rep 35, 109105, (2021).
Riggan, L. et al. CRISPR-Cas9 Ribonucleoprotein-Mediated Genomic Editing in Mature Primary Innate Immune Cells. Cell Rep 31, 107651, (2020).
DeWitt, M. A., Corn, J. E. & Carroll, D. Genome editing via delivery of Cas9 ribonucleoprotein. Methods 121-122, 9-15, (2017).
Bae, S., Park, J. & Kim, J. S. Cas-OFFinder: a fast and versatile algorithm that searches for potential off-target sites of Cas9 RNA-guided endonucleases. Bioinformatics 30, 1473-1475, (2014).
Bergen, V., Lange, M., Peidli, S., Wolf, F. A. & Theis, F. J. Generalizing RNA velocity to transient cell states through dynamical modeling. Nat Biotechnol 38, 1408-1414, (2020).
Gulati, G. S. et al. Single-cell transcriptional diversity is a hallmark of developmental potential. Science 367, 405-411, (2020).
Gerstner, N. et al. GeneTrail 3: advanced high-throughput enrichment analysis. Nucleic Acids Res 48, W515-W520, (2020).
Garg, R. K. et al. Capillary force seeding of hydrogels for adipose-derived stem cell delivery in wounds. Stem Cells Transl Med 3, 1079-1089, (2014).
Rustad, K. C. et al. Enhancement of mesenchymal stem cell angiogenic capacity and stemness by a biomimetic hydrogel scaffold. Biomaterials 33, 80-90, (2012).
Wong, V. W. et al. Pullulan hydrogels improve mesenchymal stem cell delivery into high-oxidative-stress wounds. Macromol Biosci 11, 1458-1466, (2011).
Michaels, J. t. et al. Wound Repair Regen 15, 665-670, (2007).
Liu, Z., Wu, H. & Huang, S. Role of NGF and its receptors in wound healing (Review). Exp Ther Med 21, 599, (2021).
Kim, M. H. et al. Wound Repair Regen 26 Suppl 1, S9-S18, (2018).
Lin, Y. T. et al. J Invest Dermatol 135, 258-268, (2015).
Jin, S. et al. Nat Commun 12, 1088, doi:10.1038/s41467-021-21246-9 (2021).
Liaw, L. et al. J Clin Invest 101, 1468-1478, doi:10.1172/JCl2131 (1998).
Sharma, A., Singh, A. K., Warren, J., Thangapazham, R. L. & Maheshwari, R. K. J Invest Dermatol 126, 2323-2331, doi:10.1038/sj.jid.5700428 (2006).
Wang, W. et al. PLoS One 12, e0185346, (2017).
Park, J. H. et al. N Engl J Med 378, 449-459, (2018).
Lundh, S. et al. Clin Exp Med 20, 469-480, (2020).
Laguette, N. et al. Nature 474, 654-657, (2011).
Breckpot, K. et al. Hum Gene Ther 18, 536-546, (2007).
Witkowski, W. et al. PLoS One 10, e0133651, (2015).
Norton, T. D., Miller, E. A., Bhardwaj, N. & Landau, N. R. Gene Ther 22, 227-236, (2015).
Wong, V. W. et al. Tissue Eng Part A 17, 631-644, (2011).
Hopewell, E. L. & Cox, C. Mol Ther Methods Clin Dev 16, 155-160, (2020).
Lutz, M. B. et al. J Immunol Methods 223, 77-92, (1999).
Donovan, D., Brown, N. J., Bishop, E. T. & Lewis, C. E. Angiogenesis 4, 113-121, (2001).
Li, H. & Durbin, R. Bioinformatics 25, 1754-1760, (2009).
Kim, S. et al. Nat Methods 15, 591-594, (2018).
Li, J. et al. J Cell Mol Med 22, 2727-2738, (2018).
Stuart, T. et al. Cell 177, 1888-1902 e1821, (2019).
Diaz-Papkovich, A., Anderson-Trocme, L., Ben-Eghan, C. & Gravel, S. PLoS Genet 15, (2019).
Mostafavi, S. et al. J Immunol 193, 4485-4496, (2014).
Aran, D. et al. Nat Immunol 20, 163-172, (2019).
Galiano, R. D., Michaels, J. t., Dobryansky, M., Levine, J. P. & Gurtner, G. C. Wound Repair Regen 12, 485-492, (2004).

1. A method of enhancing wound healing in an individual, comprising:
contacting a cutaneous wound with a matrix or scaffold film comprising an effective dose of an edited dendritic cell (DCs) composition localized to the site of the wound by the matrix or scaffold, for a period of time sufficient to enhance healing.

2. The method of claim 1, wherein the edited DC are genetically manipulated to have reduced N-myc downregulated gene 2 (Ndrg2) expression.

3. The method of claim 1, wherein the edited DC are genetically manipulated to knock out the Ndrg2 gene (Ndrg2 KO).

4. The method of claim 1, wherein the matrix or scaffold film is a hydrogel film.

5. The method of claim 1, wherein the individual is a human.

6. The method of claim 1, wherein the individual has diabetes.

7. The method of claim 1, wherein the matrix is a pullulan-collagen composite hydrogel.

8. The method of claim 1, wherein the edited DCs have increased expression of one more genes selected from Vascular endothelial growth factor (Vegfa), Fibronectin-1 (Fn1), Matrix metalloproteinase-12 (Mmp12), Peroxiredoxin 4 (Prdx4), and Microsomal Glutathione S-Transferase 1 (Mgst1) relative to DCs that have not been edited.

9. The method of claim 1, wherein the effective dose of edited DC is from about 10³ cells/mm² of hydrogel, up to about 10⁶ cells/mm² of hydrogel film.

10. The method of claim 9, wherein the effective dose of edited DCs is from about 10⁴ to about 10⁵ cells/cm² of hydrogel.

11. The method of claim 1, wherein the cell composition is autologous relative to the individual.

12. The method of claim 1, wherein the cell composition is allogeneic relative to the individual.

13. The method of claim 1, wherein the wound is a chronic wound.

14. The method of claim 1, wherein the effective amount results in increased vascularization in the wound bed relative to no treatment.

15. A method of producing a therapeutic dendritic cell having reduced or no Ndrg2 expression (Ndrg2 KO DC), the method comprising:
contacting a dendritic cell with a Cas9/single guide RNA-ribonucleoprotein complex (Cas9-RNP) comprising one or more single guide RNAs (sgRNAs).

16. The method of claim 15, wherein the sgRNA comprises more than 12 base pairs complimentary to the gene-coding sequence of Ndrg2 or Ndrg2's regulatory elements

17. The method of claim 15, wherein the Cas9-RNP comprises three or more sgRNAs.

18. The method of claim 15, wherein the sgRNA has a sequence selected from SEQ ID NO: 4, 5 and 6.

19. The method of claim 15, wherein Cas9-RNP comprises three sgRNAs having sequences corresponding to SEQ ID NO: 1-3.

20. A hydrogel-cell composition comprising a pullulan-collagen hydrogel gel film and the Ndrg2 KO DC of claim 15.


SEQUENCE LISTING (40 sequences)

SEQ ID NO: 1 - DNA, 20 bp, synthetic construct
  source 1..20: mol_type = other DNA
gtaaaagtgaccgagccata

SEQ ID NO: 2 - DNA, 20 bp, synthetic construct
  source 1..20: mol_type = other DNA
ccagccactgtttcggttcg

SEQ ID NO: 3 - DNA, 20 bp, synthetic construct
  source 1..20: mol_type = other DNA
acaataattggagttggtgt

SEQ ID NO: 4 - DNA, 20 bp, synthetic construct
  source 1..20: mol_type = other DNA
atgttcagagcatgggaccg

SEQ ID NO: 5 - DNA, 20 bp, synthetic construct
  source 1..20: mol_type = other DNA
gcagtctcgggtgtttgtcc

SEQ ID NO: 6 - DNA, 20 bp, synthetic construct
  source 1..20: mol_type = other DNA
ctcctgaagttctgccatgg

SEQ ID NO: 7 - DNA, 20 bp, synthetic construct
  source 1..20: mol_type = other DNA
cgttaatcgcgtataatacg

SEQ ID NO: 8 - DNA, 20 bp, synthetic construct
  source 1..20: mol_type = other DNA
catattgcgcgtatagtcgc

SEQ ID NO: 9 - DNA, 20 bp, synthetic construct
  source 1..20: mol_type = other DNA
ggcgcgtatagtcgcgcgta

SEQ ID NO: 10 - DNA, 25 bp, synthetic construct
  source 1..25: mol_type = other DNA
tctgcatccctcccatttatcctct

SEQ ID NO: 11 - DNA, 25 bp, synthetic construct
  source 1..25: mol_type = other DNA
cagaacccaccctcttgctctgatg

SEQ ID NO: 12 - DNA, 27 bp, synthetic construct
  source 1..27: mol_type = other DNA
ccatgatgtaggcctcaactgtaagac

SEQ ID NO: 13 - DNA, 23 bp, synthetic construct
  source 1..23: mol_type = other DNA
tcttctctggagcaacctccgca

SEQ ID NO: 14 - DNA, 25 bp, synthetic construct
  source 1..25: mol_type = other DNA
cagagagccgagaattgataaactc

SEQ ID NO: 15 - DNA, 25 bp, synthetic construct
  source 1..25: mol_type = other DNA
gcaggaactcggaccattcctgcaa

SEQ ID NO: 16 - DNA, 26 bp, synthetic construct
  source 1..26: mol_type = other DNA
ctggtgttgctctgcaccttgcattg

SEQ ID NO: 17 - DNA, 25 bp, synthetic construct
  source 1..25: mol_type = other DNA
tggttacctgtccctggtccaggag

SEQ ID NO: 18 - DNA, 23 bp, synthetic construct
  source 1..23: mol_type = other DNA
ccagaagaaacaagtgagatgct

SEQ ID NO: 19 - DNA, 24 bp, synthetic construct
  source 1..24: mol_type = other DNA
atgtgtagtagtatgggacctggg

SEQ ID NO: 20 - DNA, 24 bp, synthetic construct
  source 1..24: mol_type = other DNA
gtgtttagtgcaatgtgactgggg

SEQ ID NO: 21 - DNA, 20 bp, synthetic construct
  source 1..20: mol_type = other DNA
gcagtctcgggtgtttgtcc

SEQ ID NO: 22 - DNA, 22 bp, synthetic construct
  source 1..22: mol_type = other DNA
ttccttgatgtgccaaggtggg

SEQ ID NO: 23 - DNA, 23 bp, synthetic construct
  source 1..23: mol_type = other DNA
cccccatggcataagcacatgag

SEQ ID NO: 24 - DNA, 24 bp, synthetic construct
  source 1..24: mol_type = other DNA
ccatgggacccatgttcttatcat

SEQ ID NO: 25 - DNA, 25 bp, synthetic construct
  source 1..25: mol_type = other DNA
atgtcctcagagcttaggacccagg

SEQ ID NO: 26 - DNA, 23 bp, synthetic construct
  source 1..23: mol_type = other DNA
atcttcagagcttggccccggaa

SEQ ID NO: 27 - DNA, 20 bp, synthetic construct
  source 1..20: mol_type = other DNA
atgttcagagcatgggaccg

SEQ ID NO: 28 - DNA, 24 bp, synthetic construct
  source 1..24: mol_type = other DNA
ccggactggctagaactacaggtg

SEQ ID NO: 29 - DNA, 23 bp, synthetic construct
  source 1..23: mol_type = other DNA
cccctccccatgccctgaataca

SEQ ID NO: 30 - DNA, 23 bp, synthetic construct
  source 1..23: mol_type = other DNA
ccactgacatgctccctacatga

SEQ ID NO: 31 - DNA, 23 bp, synthetic construct
  source 1..23: mol_type = other DNA
ccccagtcccatctctgaaagcc

SEQ ID NO: 32 - DNA, 23 bp, synthetic construct
  source 1..23: mol_type = other DNA
cctccttccctctctctgaactc

SEQ ID NO: 33 - DNA, 23 bp, synthetic construct
  source 1..23: mol_type = other DNA
cctctctctctctctgaacatgg

SEQ ID NO: 34 - DNA, 23 bp, synthetic construct
  source 1..23: mol_type = other DNA
cctccataggatcgtgaggataa

SEQ ID NO: 35 - DNA, 25 bp, synthetic construct
  source 1..25: mol_type = other DNA
cctaggttccatgacctctggagaa

SEQ ID NO: 36 - DNA, 23 bp, synthetic construct
  source 1..23: mol_type = other DNA
ccctgaaacacccacaactgtct

SEQ ID NO: 37 - DNA, 25 bp, synthetic construct
  source 1..25: mol_type = other DNA
ctacattggagttcattcatggagg

SEQ ID NO: 38 - DNA, 23 bp, synthetic construct
  source 1..23: mol_type = other DNA
ctactgaaagcctgccctggagg

SEQ ID NO: 39 - DNA, 20 bp, synthetic construct
  source 1..20: mol_type = other DNA
ctcctgaagttctgccatgg

SEQ ID NO: 40 - DNA, 130 bp, synthetic construct
  source 1..130: mol_type = other DNA
gccaccatggcagaacttcaggaggtgcagatcaccgaggagaagccgcttgccaggacaaacacccgagactgccaaggtttacagagactctctgacttccctgcctcggtcccatgctctgaacatc